(19) United States
(12) Patent Application Publication
(10) Pub. No.: US 2022/0313651 A1
(43) Pub. Date: Oct. 6, 2022

(54) INHIBITORS OF ZIKA VIRUS INFECTION

(71) Applicant: New York Blood Center, Inc., New York, NY (US)

(72) Inventors: Lanying DU, New York, NY (US); Asim Kumar DEBNATH, New York, NY (US); Shibo JIANG, New York, NY (US); Yaning GAO, New York, NY (US)

(21) Appl. No.: 17/636,775

(22) PCT Filed: Aug. 20, 2020
(86) PCT No.: PCT/US2020/047233

§ 371 Date: Feb. 18, 2022

Related U.S. Application Data
(60) Provisional application No. 62/889,246, filed on Aug. 20, 2019.

(51) Int. Cl. A61K 31/343; A61K 31/7048; A61K 31/58; A61K 31/137; A61K 31/4184; A61K 31/165; A61K 31/4196; A61K 31/12; A61K 31/69; A61P 31/14; A61K 31/11

(52) U.S. Cl. CPC A61K 31/343; A61K 31/7048; A61K 31/58; A61K 31/137; A61K 31/4184; A61K 31/165; A61K 31/4196; A61K 31/12; A61K 31/69; A61P 31/14; A61K 31/11

(57) ABSTRACT

The present specification provides methods of treatment of flavivirus infection, such as Zika or dengue vims infection, by administration of gossypol, a gossypol derivative, digitonin, or conessine. These compounds may be used alone, in combination with each other, or in combination with curcumin or bortezomib.

### CROSS REFERENCE TO RELATED APPLICATIONS

The present application claims the benefit of U.S. Provisional Patent Application 62/889,246 filed Aug. 20, 2020, the entire contents of which is incorporated by reference herein.

### BACKGROUND

Zika virus (ZIKV) is a mosquito-borne flavivirus in the same genus as other important human pathogens, including dengue virus (DENV), West Nile virus (WNV), yellow fever virus (YFV), Japanese encephalitis virus (JEV), and tick-borne encephalitis virus (TBEV). ZIKV was originally isolated in a rhesus macaque in 1947, but this virus has only recently got worldwide attention owing to its close association with congenital Zika syndrome (CZS), as represented by microcephaly, fetal demise, central nervous system abnormalities, and other neurological complications. No antiviral therapeutics for the treatment of ZIKV-associated human diseases, particularly congenital syndrome and fetal death, have been approved.

### SUMMARY

Disclosed herein are methods of treating a flavivirus infection, particularly a Zika virus infection. Other embodiments are methods of treating other flavivirus infections, for example mosquito-borne and tick-borne flavivirus (TBFV) infections. Mosquito-borne flaviviruses infections include infections by DENV, WNV, JEV, and YFV, for example. TBFV infections include infections by TBEV, Powassan virus, and Langat virus, for example. Some embodiments comprise inhibiting a flavivirus infection.

These treatments comprise administration of gossypol, a gossypol derivative, digitonin, or conessine to a person in need thereof. In some embodiments, treatment includes administration of at least a second active agent. In an aspect of such embodiments, enhanced or synergistic effect is achieved. The second agent can comprise gossypol, a gosspol derivative, digitonin, conessine, curcumin, or bortezomib (as long as the first and second active agent are not the same).

In some embodiments the treatment or inhibition is therapeutic. In some embodiments the treatment or inhibition is prophylactic.

The above compounds, gossypol, ST069299, ST005138, ST092971, ST086276, or ST087010, curcumin, digitonin, conessine, and bortezomib, derivatives thereof, and compositions, including pharmaceutical compositions, comprising them individually or in any combination, shall be referred to herein collectively as Z-medicaments or test compounds.

Disclosed herein are methods of treating a flavivirus infection, comprising administering an effective amount of gossypol, digitonin, conessine, ST069299, ST005138, ST092971, ST086276, or ST087010 to a person in need thereof.

Also disclosed herein are methods of inhibiting a flavivirus infection, comprising administering an effective amount of gossypol, digitonin, conessine, ST069299, ST005138, ST092971, ST086276, or ST087010 to a person in need thereof.

In some embodiments, the inhibiting is prophylactic. In some embodiments, the administering of gossypol, digitonin, conessine, ST069299, ST005138, ST092971, ST086276, or ST087010 takes place within 12 hours prior to potential exposure.

In some embodiments, the inhibiting is therapeutic. In some embodiments, the administering of gossypol, digitonin, conessine, ST069299, ST005138, ST092971, ST086276, or ST087010 takes place within 24 hours after exposure or potential exposure.

In some embodiments, the methods comprise administration of gossypol, ST069299, ST005138, ST092971, ST086276, or ST087010. In some embodiment, the methods further comprise administration of digitonin or conessine.

In some embodiments, the methods comprise administration of ST069299. In some embodiment, the methods further comprise administration of digitonin or conessine. In some embodiments, the methods comprise administration of ST005138. In some embodiment, the methods further comprise administration of digitonin or conessine.

In some embodiments, the methods comprise administration of ST092971. In some embodiment, the methods further comprise administration of digitonin or conessine.

In some embodiments, the methods comprise administration of ST086276. In some embodiment, the methods further comprise administration of digitonin or conessine.

In some embodiments, the methods comprise administration of ST087010. In some embodiment, the methods further comprise administration of digitonin or conessine.

In some embodiments, the methods comprise administration of digitonin. In some embodiment, the methods further comprise administration of gossypol, conessine, ST069299, ST005138, ST092971, ST086276, or ST087010.

In some embodiments, the methods comprise administration of conessine. In some embodiment, the methods further comprise administration of gossypol, digitonin, ST069299, ST005138, ST092971, ST086276, or ST087010.

In some embodiments, the methods further comprise administration of curcumin and/or bortezomib.

In some embodiments, the flavivirus is a Spondweni virus. In some embodiments, the Spondweni virus is Zika virus. In some embodiments, the flavivirus is a dengue virus. In some embodiments, the flavivirus is a Japanese encephalitis group virus. In some embodiments, the flavivirus is a yellow fever virus group virus. In some embodiments, the flavivirus is a mosquito-borne human virus.

In some embodiments, the person in need thereof is infected with the flavivirus. In some embodiments, the person in need thereof has been exposed to the flavivirus. In some embodiments, the person in need thereof is at risk of exposure to the flavivirus.

### BRIEF DESCRIPTION OF DRAWINGS

FIG. 1 depicts the structure of four compounds, gossypol, curcumin, digitonin, and conessine which were identified as inhibitors against ZIKV (strain PAN2016 (2016/Panama)).

FIG. 2A-K depicts a time-of-addition experiment to test the ability of the disclosed compounds to block ZIKV infection at different steps of the viral life cycle. The time-of-addition experiments were performed in Vero E6 cells, and specific procedures are illustrated in detail in FIG. 2A-F. FIG. 2A: pretreatment of ZIKV: ZIKV was incubated with one of the compounds (gossypol, curcumin, digitonin, or conessine) or an anti-ZIKV compound control (bortezomib), at 37° C. for 1 h. After removal of the unbound compounds, the treated ZIKV was incubated with cells at 37° C. for 1 h, followed by culture of the cells at 37° C. for 4-5 days before enumeration of plaques and calculation of inhibition rate. FIG. 2B: Pretreatment of cells: cells were pre-incubated with one of the compounds at 37° C. for 1 h, and the unbound compounds were then removed, followed by addition of ZIKV and incubation of cells at 37° C. for 1 h. After removal of the unbound ZIKV, the cells were cultured and plaques and inhibition rate determined as in FIG. 2A. FIG. 2C: Blockage of ZIKV attachment: cells were incubated with ZIKV at 4° C. for 1 h (to allow ZIKV attachment but not fusion between ZIKV and cell membranes) in the presence of one of the compounds. After removal of the unbound ZIKV and compounds, the cells were cultured and plaques and inhibition rate determined. FIG. 2D: Co-treatment of ZIKV and cells: cells were infected with ZIKV at 37° C. for 1 h in the presence of one of the compounds, followed by removal of the unbound viruses and compounds, and culture of the cells to determine plaques and inhibition rate. FIG. 2E: Blockage of ZIKV penetration (membrane fusion): cells were incubated with ZIKV at 4° C. for 1 h to allow ZIKV attachment. After removal of the unbound ZIKV, the cells were incubated with one of the compounds at 37° C. for 1 h to allow fusion of virus-cell membranes. After further removal of the unbound compounds, the cells were cultured and plaques and inhibition rate determined. FIG. 2F: Inhibition of post-entry stage: cells were incubated with ZIKV at 37° C. for 1 h (to allow ZIKV entry into the target cells). After removal of the unbound ZIKV, the cells were further incubated with one of the compounds at 37° C. for 1 h, followed by removal of the unbound compounds and culture of cells for determination of plaques and inhibition rates. Inhibition of ZIKV infection by gossypol (FIG. 2G), curcumin (FIG. 2H), digitonin (FIG. 2I), and conessine (FIG. 2J), as well as anti-ZIKV compound control (bortezomib) (FIG. 2K), was assayed against ZIKV (PAN2016) infection using the above six steps. The percent inhibition was calculated based on the numbers of plaques in the presence or absence of serially diluted compounds. The data are expressed as mean±s.e.m. (n=2). The experiments were repeated three times with similar results.

FIG. 3A-G depicts the binding of the disclosed compounds to ZIKV proteins and inhibition of the binding of ZIKV envelope (E) protein domain III (EDIII) to ZIKV EDIII-specific monoclonal antibodies (mAbs), as well as ZIKV non-structure protein (NS2B-NS3) protease activity. Binding of compounds (NPs) to ZIKV full-length E (FIG. 3A), EDIII (FIG. 3B), and NS2B-NS3 (FIG. 3C) proteins, was detected by ELISA. The percent binding was reported in the presence or absence of serially diluted NPs using the formula (1-[E/EDIII-NP]/[E/EDIII])×100 (for E/EDIII binding) or (1-[NS2B-NS3-NP]/[NS2B-NS3])×100 (for NSEB-NS3 binding). 50% effective concentration (EC₅₀) values were calculated based on the percent binding using the CalcuSyn computer program. Surface Plasmon Resonance (SPR) analysis of binding between gossypol and ZIKV E protein (FIG. 3D) or NS2B-NS3 protein (FIG. 3E) was measured. Binding affinity (K_{D}: equilibrium dissociation constant) is shown in each figure. FIG. 3F depicts the ability of gossypol to inhibit the binding between ZIKV EDIII and EDIII-specific neutralizing mAbs. The concentrations of ZIKV EDIII and mAbs were 1.5 and 0.5 μg/ml, respectively. The percent inhibition in the EDIII-mAb binding was measured in the presence or absence of serially diluted gossypol using the formula (1-[EDIII-mAb-gossypol]/[EDIII-mAb])×100, which, in turn, formed the basis for calculating 50% inhibitory concentration (IC₅₀) values. FIG. 4G depicts the ability of the disclosed compounds to inhibit ZIKV NS2B-NS3 protease activity. The concentrations of substrate (Bz-Nle-Lys-Lys-Arg-AMC) and ZIKV NS2B-NS3 protein were 4 μM and 1 μg/ml, respectively. The percent inhibition of protease activity was calculated in the presence or absence of serially diluted compounds using the formula (1-[NS2B-NS3-substrate-NP]/[NS2B-NS3-substrate])×100. The IC₅₀ values were calculated based on the percent inhibition. The data are expressed as mean±s.e.m. (n=4). Bortezomib, a previously reported anti-ZIKV inhibitor, was used as an anti-ZIKV control, and DMSO was included as a negative control. The experiments were repeated twice with similar results.

FIG. 4A-B depicts structure, anti-Zika virus (ZIKV) activity, and cytotoxicity of gossypol derivatives. The experiments were performed on Vero E6 cells, and the cytotoxicity of gossypol derivatives in this cell line is expressed as 50% cytotoxic concentration (CC₅₀). The inhibitory activity of gossypol derivatives against infection of ZIKV human strain PAN2016 (2016/Panama) is expressed as 50% inhibitory concentration (IC₅₀). Selectivity index (SI) was calculated using formula (CC₅₀/IC₅₀). IC₅₀, CC₅₀, and SI values of gossypol and each of its derivatives are shown. The circled substituents (FIG. 4A) are aldehyde groups at the C8 and C8′ positions of gossypol which are replaced by other groups, whereas the gray shading (FIG. 4B) shows that the free hydroxyl groups at the C7 and CT positions of gossypol core have been changed to carbonyl oxygens. The data are presented as the mean±standard error of the mean (s.e.m.) (n=2). The experiments were repeated twice with similar results.

FIG. 5 depicts potential inhibitory mechanism of gossypol and its derivative, ST087010, against ZIKV infection. Time-of-addition experiments were performed in Vero E6 cells, and six specific steps are shown as follows. 1) Step 1: Pretreatment of virus. ZIKV was incubated with compounds gossypol or ST087010 at 37° C. for 1 h. After removal of the unbound compounds, ZIKV was incubated with cells at 37° C. for 1 h, followed by culturing cells at 37° C. for 4-5 days, and enumerating plaques. 2) Step 2: Pretreatment of cells. Cells were preincubated with gossypol or ST087010 at 37° C. for 1 h, and the unbound compounds were removed, followed by addition of ZIKV and incubation of cells at 37° C. for 1 h. The unbound ZIKV was removed, and the cells were cultured and plaques enumerated as in Step 1. 3) Step 3: Attachment. Cells were incubated with ZIKV at 4° C. for 1 h in the presence of gossypol or ST087010, which will allow ZIKV attachment, but not ZIKV and cell membrane fusion. After removal of the unbound ZIKV and compounds, the cells were cultured and plaques enumerated as in Step 1. 4) Step 4: Co-treatment. Cells were infected with ZIKV at 37° C. for 1 h in the presence of gossypol or ST087010. After removal of the unbound viruses and compounds, the cells were cultured and plaques enumerated as in Step 1. 5) Step 5: Fusion. Cells were incubated with ZIKV at 4° C. for 1 h for ZIKV attachment. After removal of the unbound ZIKV, the cells were incubated with gossypol or ST087010 at 37° C. for 1 h for virus-cell membrane fusion. The unbound compounds were removed, and the cells were cultured and plaques enumerated as in Step 1. 6) Step 6: Post-entry. Cells were incubated with ZIKV at 37° C. for 1 h for ZIKV entry. After removal of the unbound ZIKV, the cells were incubated with gossypol or ST087010 at 37° C. for 1 h. The unbound compounds were then removed, and the cells were cultured and enumerated for plaques as in Step 1. The data are expressed as mean % inhibition±s.e.m. (n=2). The percent inhibition was calculated based on the numbers of plaques in the presence or absence of the compounds. The experiments were repeated three times with similar results.

FIG. 6A-E depicts binding affinity of gossypol and its derivative, ST087010, to ZIKV proteins. Binding of ST087010 or gossypol control to ZIKV full-length envelope (E) protein (FIG. 6A), domain III of E (EDIII) protein (FIG. 6B), or NS2B-NS3 (FIG. 6C) protein was detected by ELISA. Percent binding (% binding) to E, EDIII, or NS2B-NS3 protein was calculated in the presence or absence of serially diluted compounds based on the formula ((1−(E/EDIII/NS2B-NS3−compound)/(E/EDIII/NS2B-NS3))×100), based on which 50% effective concentration (EC₅₀) was calculated. The data are expressed as mean±s.e.m. (n=4), and DMSO was used as negative control. Surface plasmon resonance (SPR) analysis of the binding between ST087010 and ZIKV EDIII (FIG. 6D) or NS2B-NS3 (FIG. 6E) protein was performed. Binding affinity was shown as K_{D} (equilibrium dissociation constant). The experiments were repeated twice with similar results.

FIG. 7A-F depicts the ability of gossypol and its derivative, ST087010, to inhibit binding of ZIKV EDIII to EDIII-specific neutralizing mAbs, as well as ZIKV NS2B-NS3 protease activity. FIG. 7A-D depicts percent inhibition (% inhibition) of the EDIII-mAb binding in the presence or absence of serially diluted compound (ST087010 or gossypol control) based on the formula ((1−(EDIII-mAb-compound)/(EDIII-mAb)×100). The concentrations of ZIKV EDIII and mAbs were 1.5 and 0.5 μg/ml, respectively. Four ZIKV EDIII-specific mAbs were used, and IC₅₀ values were calculated. ZIKV EDI/DU-specific mAb ZKA78 (FIG. 7E) and DMSO were used as controls. FIG. 7F depicts the ability of ST087010 in inhibition of ZIKV NS2B-NS3 protease activity. The concentrations of substrate (Bz-Nle-Lys-Lys-Arg-AMC) and ZIKV NS2B-NS3 protein were 4 μM and 1 μg/ml, respectively. Percent inhibition (% inhibition) of protease activity was measured in the presence or absence of serially diluted compounds and calculated based on the formula ((1−(NS2B-NS3-substrate-compound)/(NS2B-NS3-substrate)×100), and IC₅₀ values were calculated. The data are expressed as mean±s.e.m. (n=4). The experiments were repeated twice with similar results.

FIG. 8A-F depicts efficacy of gossypol and its derivative, ST087010, in protecting Ifnar1^{-/-} mice from lethal ZIKV challenge. FIG. 8A is a schematic diagram of experimental procedures. Six-to-seven-week-old male mice were intraperitoneally (i.p.) treated with gossypol derivative ST087010 or gossypol (as a control) (20 mg/kg), as well as DMSO (negative control), 12 h before and 6, 24 and 48 h after infection. The treated mice were infected with ZIKV human strain R103451 (200 plaque forming unit (PFU)/mouse), followed by evaluation of survival rate (FIG. 8B) or weight changes (FIG. 8C) for 21 days. The data are expressed as mean±s.e.m. of mice in each group (n=6). In a separate experiment, ST087010 or gossypol-treated mice were infected with ZIKV human strain PAN2016 (200, PFU/mouse). Five days post-infection (dpi), viral titers were detected in tissues by plaque assay (FIG. 8D), and ZIKV or caspase-3 signals were measured in eye (FIG. 8E) and testis (FIG. 8G) tissues by immunofluorescence staining. The data are expressed as mean±s.e.m. of mice in each group (n=5), and significant differences among different groups are shown as *, **, and ***. The detection limit is 25 PFU/g. ZIKV and caspase-3 (in FIG. 8E-F) were stained with anti-ZIKV and anti-active caspase-3 antibodies, respectively. Nuclei were stained with DAPI (40,6-diamidino-2-phenylindole). Representative images of immunofluorescence staining are shown. Magnification, 63×, and scale bar, 10 μm.

FIG. 9A-H depicts the efficacy of gossypol derivative ST087010 in protecting pregnant Ifnar1^{-/-} mice and their fetuses against ZIKV challenge. Pregnant mice (at embryonic day (E) 12-14) were i.p. treated with ST087010 (20 mg/kg) or DMSO (control) 12 h before and 6, 24 and 48 after infection with ZIKV human strain R116265 (10³ PFU/mouse). Viral titers were detected, by plaque assay, in sera (FIG. 9A), placenta (FIG. 9B), fetal brain (FIG. 9C), and amniotic fluid (FIG. 9D) at 5 days post infection (dpi). The data are expressed as mean±s.e.m. of mice in each group (n=5), and significant differences among different groups are shown. The detection limit is 25 PFU/ml (for sera and amniotic fluid), or 25 PFU/g (for placenta and fetal brain). Representative images of morphology of mouse uteri and fetuses at 5 dpi are shown. FIG. 9E depicts E17-19 uteri from pregnant mice challenged at E12-14. Fetal morphology (FIG. 9F) and fetal size (FIG. 9G) were detected. ZIKV or caspase-3 signals were measured in placentas by immunofluorescence staining (FIG. 9H). ZIKV and caspase-3 were stained with anti-ZIKV and anti-active caspase-3 antibodies, respectively. Nuclei were stained with DAPI. Representative images of immunofluorescence staining are shown. Magnification, 63×, and scale bar 10 μm.

FIG. 10A-E depicts the safety profile of gossypol derivative ST087010 in pregnant Ifnar1^{-/-} mice and their pups. FIG. 10A depicts weight changes of pregnant mothers at prenatal and postnatal time points and FIG. 10B depicts weight changes of pups at different postnatal time points. Alanine aminotransferase (ALT) (FIG. 10C) and creatinine (FIG. 10D) levels in sera of pregnant mice were measured by ALT assay and Creatinine assay, respectively, before and 4 h, 1, 3 and 5 days post-last injection of ST087010 or DMSO control. Haematoxylin and eosin (H&E) staining of tissues (FIG. 10E), including liver, spleen, kidney, and brain, from ST087010 or DMSO-treated mothers and their pups. Scale bar, 50 μm.

FIG. 11A-C depicts the efficacy of gossypol and its derivative, ST087010, in protecting Ifnar1^{-/-} mice from DENV-2 challenge. FIG. 11A is a schematic diagram of experimental procedures. Three-to-four-week-old male mice were treated with i.p injection of ST087010 or gossypol control (20 mg/kg), or DMSO (negative control) 12 h before and 6, 24, and 48 h after infection with DENV-2 human strain V594 (2×10⁶ PFU/mouse). FIG. 11B depicts representative images of DENV titers in brain, kidney, heart, and sera analyzed by flow cytometry at 3 dpi. C6/36 cells with or without DENV-2 infection were used as positive and negative controls, respectively. The percentage of infected cells to the total number of cells is shown in each figure. FIG. 11C depicts the detection of DENV titers in challenged mouse tissues and sera at 3 dpi. Viral titers are expressed as infectious units/g (for brain, kidney, or heart), or infectious units/ml (for sera), as detected by flow cytometry and calculated based on (B). *, **, and *** indicate significant differences of DENV infection between ST087010 and gossypol or DMSO groups, or between gossypol and DMSO groups. The data are presented as mean±s.e.m of duplicate wells (n=2). The experiments were repeated twice with similar results.

### DETAILED DESCRIPTION

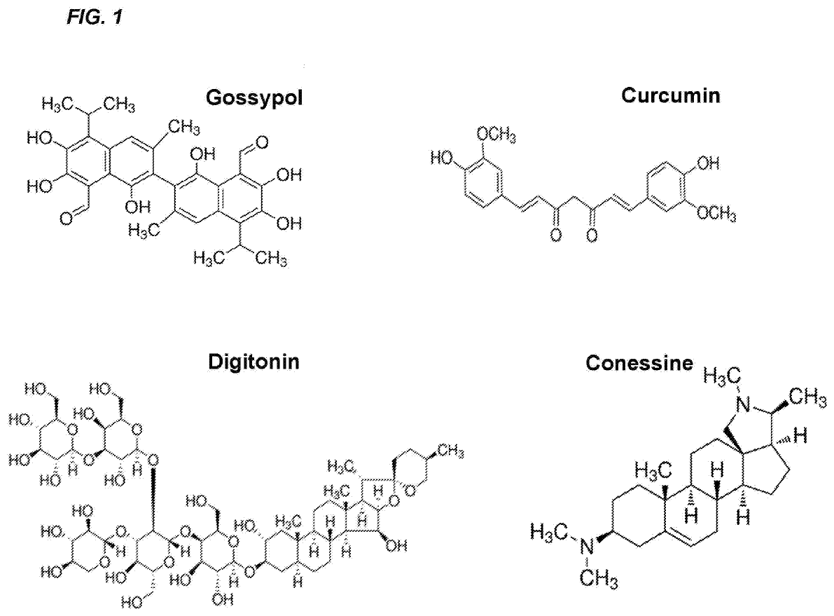

Zika virus (ZIKV) infection during pregnancy leads to severe congenital Zika syndrome, including microcephaly and other neurological malformations. No therapeutic agents have, to date, been approved for the treatment of ZIKV infection in humans, despite a need for effective and safe antiviral drugs to treat ZIKV-caused diseases. After screening a compound library, lead compounds were identified with anti-ZIKV activity in Vero E6 cells: gossypol, curcumin, digitonin, and conessine (FIG. 1). Anti-ZIKV activity has been reported previously for curcumin, but the anti-ZIKV activity of gossypol, digitonin, and conessine was not previously known. Among them, gossypol exhibited the strongest inhibitory potency against almost all ten ZIKV strains tested, including six recent epidemic human strains. The mechanistic study indicated that gossypol could neutralize ZIKV infection by targeting the envelope protein domain III (EDIII) and NS2B-NS3 protease of ZIKV. In contrast, the other compounds inhibited ZIKV infection by targeting the host cell or cell-associated entry and replication stages of ZIKV infection. Combination of gossypol with any of the three compounds identified in this study, and as well as with the proteasome inhibitor bortezomib, a previously reported anti-ZIKV compound, exhibited significant synergistic inhibitory effects against three ZIKV human strains tested. Particularly, gossypol derivative ST087010 presented broad-spectrum in vitro and in vivo activity against multiple ZIKV infection.

The compounds disclosed herein are collectively referred to as Z-medicaments or compounds interchangeably.

Additionally, gossypol, particularly its derivative ST087010, also demonstrated marked potency against all four serotypes of dengue virus (DENV) human strains in vitro. In addition, gossypol derivative ST087010 also presented protection against DENV-2 infection in animal models in vivo.

Taken together, these observations support use of these Z-medicaments, particularly gossypol and derivatives thereof, alone or in combination, with bortezomib and/or these other Z-medicaments, as effective broad-spectrum inhibitors against ZIKV, DENV, and other flaviviruses. Both the Spondweni viruses (including Zika virus) and the Dengue viruses are mosquito-borne flaviviruses. Other mosquito-borne, human flaviviruses include the Japanese encephalitis virus group (including Japanese encephalitis virus, St. Louis encephalitis virus, and West Nile virus) and the Yellow fever virus group (including yellow fever virus).

Gossypol is an orally-active, polyphenolic aldehyde compound that can be obtained from the cotton plant (for example, Gossypium hirsutum) or chemically synthesized. Large quantities can be obtained by extraction from cottonseed oil, though the compound is found in the stem and root of the plant as well. Gossypol permeates cells and inhibits several dehydrogenase enzymes. Gossypol has been used as an oral male contraceptive in China (at 15-20 mg/day for the 1st 3-4 months and 7.5-10 mg/day thereafter). Gossypol also has potential antineoplastic activity, inducing cell cycle arrest at the G0/G1 phase, thereby inhibiting DNA replication and leading to apoptosis, and has been used in clinical trials for the treatment of non-small cell lung cancer, non-Hodgkin lymphoma, and breast and prostate cancer. In mouse cancer models, gossypol has been used at 30-50 mg/kg/day. Antiviral activity against HIV-1, tobacco mosaic virus, herpes simplex virus II, and H5N1 influenza virus has also been reported, as have antibacterial and antiprotozoal activity.

Conessine is a steroid alkaloid. It can be isolated from plant species in the family Apocynaceae. It is known as a potent and specific antagonist of histamine H3 receptor, also has high affinity for adrenergic receptors, and has been used in treatment of malaria and as an antibacterial agent. The physiologic effect of conessine are not well understood, but it can act as an inhibitor of autophagic flux.

Digitonin is a steroidal saponin and can be obtained from the foxglove plant (Digitalis purpurea). It is effective as a weak, nonionic detergent in solubilizing lipids in water. It is commonly used a laboratory reagent to permeabilize or solubilize cell membranes. It has been reported to reverse multidrug resistance in cancer cells and to disrupt mitochondrial membranes. It has no cardiac effects and should not be confused with the cardiac drugs digoxin and digitoxin, which are also obtained from foxglove.

Curcumin is the principal curcuminoid found in the rhizomes of turmeric (Curcuma longa), and is a diarylheptanioid, diferuloylmethane. Curcumin has antioxidant activity and has been suggested to be useful as an anti-inflammatory and anti-cancer agent. Much laboratory and clinical research has been conducted on curcumin, but its medical usefulness has yet to be demonstrated and there is substantial skepticism that it will ever be.

Bortezomib is an N-protected dipeptide. It is an approved and marketed drug for multiple myeloma and mantle cell lymphoma and is the first proteasome inhibitor approved for use in humans.

The genome of ZIKV encodes a polyprotein, which is then cleaved by cellular and viral proteases to form three structural proteins, including capsid (C), precursor of membrane/membrane (prM/M), and envelope (E), as well as seven nonstructural proteins, including NS1, NS2A, NS2B, NS3, NS4A, NS4B, and NS5. The life cycle of ZIKV involves several crucial steps, including viral attachment to target cell receptor(s) or co-factors, receptor-mediated endocytosis (viral entry), virus-endosomal membrane fusion, and post-entry/post-translation stages. In this life cycle, E protein plays a key role in viral entry into target cells and subsequent fusion of virus and cell membranes, whereas NS2B and NS3 proteins consist of an important viral protease essential for post-entry/post-translational polyprotein processing, such as viral RNA replication, virion assembly, and virion release, thus, ZIKV E protein and NS2B-NS3 protease serve as therapeutic targets against ZIKV infection.

In addition to ZIKV, other flaviviruses, such as DENV, also cause significant disease in humans. Four antigenic serotypes of DENV (DENV-1-4) lead to dengue disease (dengue fever, dengue hemorrhagic fever, or dengue shock syndrome) with annually increasing cases. Therefore, the development of broad-spectrum antiviral inhibitors will be useful for the treatment of infections caused by ZIKV and other flaviviruses, including DENV. There are some important sequence similarities among proteins (such as E and NS2B-NS3) of ZIKV and other flaviviruses, such as DENV, providing the feasibility for identification of broad-spectrum anti-flavivirus inhibitors targeting these conserved sequences.

Four anti-ZIKV inhibitors have been identified by screening a compound library in cell culture, three of which (gossypol, digitonin, and conessine) have not been reported previously to have activity against ZIKV infection. While the various strains of ZIKV have nearly identical sequence, including in the EDIII domain, the Dengue virus types show a substantial degree of sequence variation from ZIKV and, to a lesser degree, amongst themselves. Thus the activity of these compounds, particularly gossypol, against the multiple ZIKV strains and all four DENV serotypes demonstrates their broad spectrum effectiveness.

Gossypol, however, is toxic, which is potentially associated with the aldehyde groups. Thus there is an urgent need to identify gossypol derivatives with potent anti-viral efficiency against ZIKV and DENV infection but low or no toxicity.

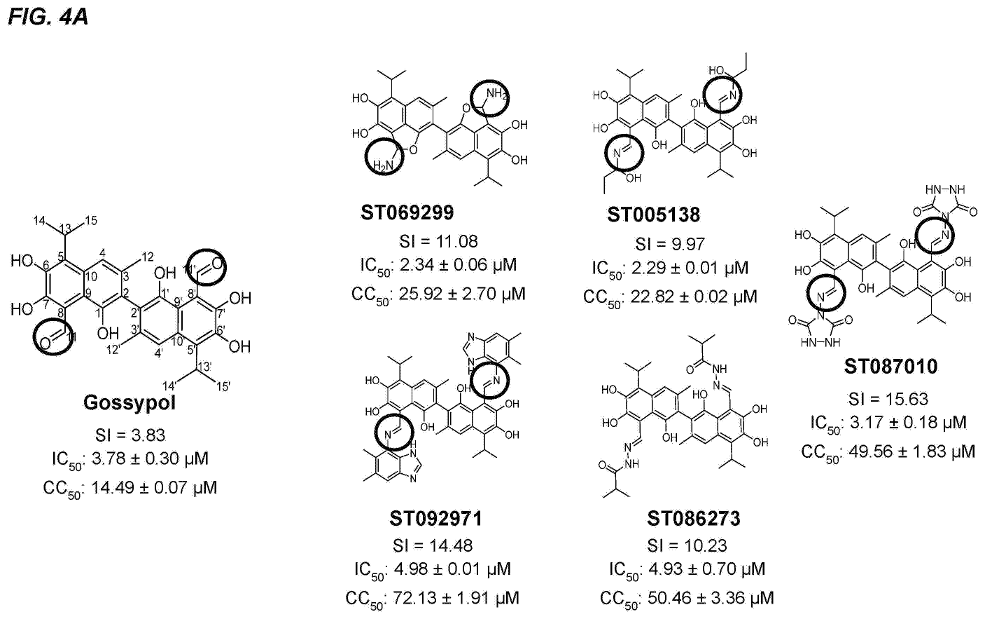

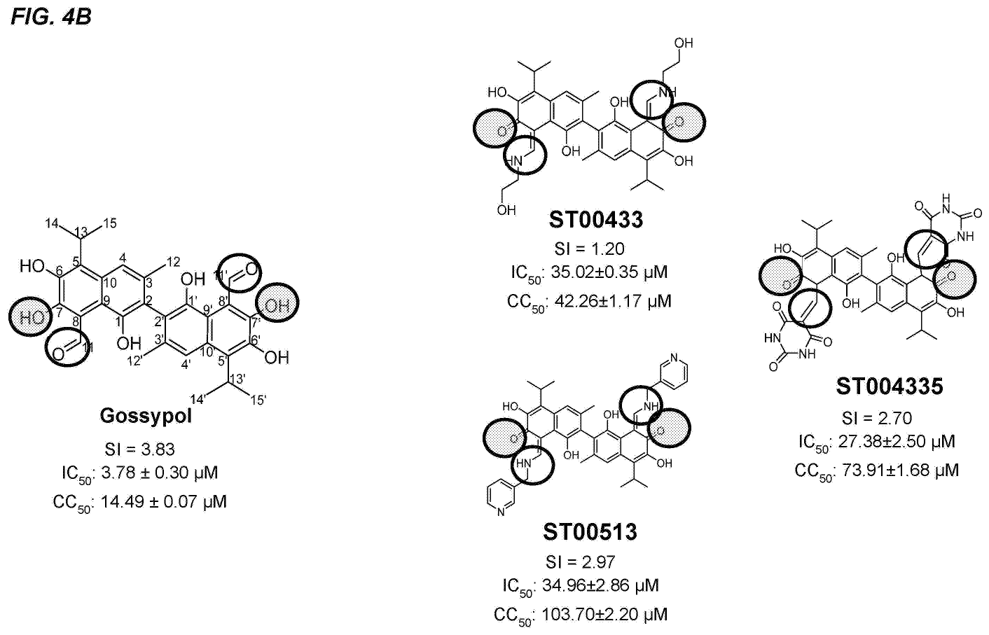

Here, a series of gossypol derivatives were screened for their anti-ZIKV and anti-DENV activity and potential cytotoxicity. Five compounds with inhibitory activity but reduced toxicity were identified (FIG. 4A-B). Among these derivatives, ST087010 demonstrated strong potency against divergent ZIKV and DENV infection, but low toxicity, and thus it was chosen for further studies for its broad-spectrum anti-ZIKV and anti-DENV activity, as well as in vivo protection against ZIKV and DENV challenge in susceptible interferon-α/β receptor (IFNAR)-deficient (Ifnar1^{-/-}) mice.

The in vitro experiments indicated that gossypol derivative ST087010 exhibited potent and broad-spectrum inhibitory activity against at least 10 ZIKV strains tested isolated from different hosts, time periods, and countries. It had significantly reduced cytotoxicity than gossypol. Similar to gossypol, ST087010 inhibited ZIKV infection by targeting the virus, rather than the other steps of virus life cycle.

The in vivo protection experiments indicated that ST087010 protected ZIKV-infected Ifnar1^{-/-} mice from mortality, which was associated with decreased viral titers in different tissues after infection of ZIKV with divergent human virus strains. In addition, ST087010 potently blocked ZIKV vertical transmission in pregnant Ifnar1^{-/-} mice, preventing ZIKV-caused fetal death. Particularly, ST087010 is safe for pregnant Ifnar1^{-/-} mice and their fetuses and pups. Moreover, ST087010 prevented infection with DENV strains 1-4 in vitro, and protected DENV-2-challenged Ifnar1^{-/-} mice against viral replication.

The mechanisms of action and/or potential targets of these compounds have been identified and revealed that they exert their effect and different stages of the infection process. This suggests that compounds can be combined for potentially synergistic effect, and indeed enhanced combinatorial effects of gossypol derivatives with other compounds in inhibiting ZIKV infection was observed.

Thus, herein disclosed are methods of treating flavivirus infection with the Z-medicament compounds disclosed herein. In particular, methods of treating flavivirus infection, wherein the Z-medicament or pharmaceutical composition comprising the Z-medicament is administered to a patient in need thereof, are disclosed. Some embodiments comprise administration of gossypol. In various aspects of these methods, the treatment further comprises administration of digitonin, conessine, curcumin, bortezomib, a gossypol derivative, or any combination thereof.

Some embodiments comprise administration of digitonin. In various aspects of these methods, the treatment further comprises administration of gossypol, conessine, curcumin, bortezomib, a gossypol derivative, or any combination thereof.

Some embodiments comprise administration of conessine. In various aspects of these methods, the treatment further comprises administration of digitonin, gossypol, curcumin, bortezomib, a gossypol derivative, or any combination thereof.

Some embodiments comprise administration of a gossypol derivative, such as ST069299, ST005138, ST092971, ST086276, or ST087010. In some embodiments, the gosspol derivative is ST087010. In various aspects of these methods, the treatment further comprises administration of digitonin, gossypol, curcumin, bortezomib, a gossypol derivative, or any combination thereof.

With respect to treatments involving multiple agents, in some embodiments, each active agent is formulated and administered separately, at the same or different times, on the same or different schedules. In other embodiments, the active agents are formulated separately, but combined shortly prior to administration and administered together. In still further embodiments, active agents are co-formulated in a single composition.

In some embodiments, the flavivirus is a mosquito-borne human flavivirus. In some embodiments the flavivirus is a Spondweni virus. In some embodiments, the Spondweni virus is a Zika virus. In some embodiments, the flavivirus is a dengue virus. In some embodiments, the dengue virus is DENV-1, DENV-2, DENV-3, or DENV-4. In some embodiments, the flavivirus is a Japanese encephalitis virus group virus. In some embodiments, the Japanese encephalitis virus subgroup virus is Japanese encephalitis virus (JEV), St. Louis encephalitis virus (SLEV), Murray Valley encephalitis virus (MVEV), or West Nile virus (WNV). In some embodiments, the flavivirus is a Yellow fever virus group virus. In some embodiments the Yellow fever virus group virus is Yellow fever virus (YFV). In some embodiments the flavivirus is a tick-borne human flavivirus, for example, TBEV, Powassan virus, and Langat virus. It should be appreciated that the classification of a virus as a human virus indicates that the virus is able to infect humans, but does not necessarily mean that it cannot infect other animal species.

The disclosed methods of treatment can be used therapeutically or prophylactically. That is, any of the Z-medicaments can be administered to an infected person to moderate the severity or duration of the infection, or symptoms thereof. In some embodiments, a Z-medicament is administered therapeutically after infection is confirmed by testing. In some embodiments, a Z-medicament is administered therapeutically after appearance of symptoms of infection. Alternatively, any of the Z-medicaments can be administered to a person prior to or just after exposure to the virus to prevent or inhibit infection or symptoms thereof, or reduce the risk of infection. In some embodiments, a Z-medicament is administered prophylactically 12 or 24 hours prior to potential exposure. In some embodiments, a Z-medicament is administered prophylactically within 2, 4, 6, 8, 12, 18, or 24 hours after exposure or suspected or possible exposure. In some cases, the Z-medicaments are administered to a person who is pregnant, in order to prevent or inhibit transmission of the infection from mother to fetus. Thus in some embodiments, treatment of the pregnant person reduces the incidence or severity of congenital Zika syndrome. As used in the present disclosure, any of the foregoing persons is “a person in need thereof”. Thus, in some embodiments, the Z-medicaments are administered to a person who has tested positive for flavivirus infection or who in exhibiting symptoms of flavivirus infection. In other embodiments, the Z-medicaments are administered to a person exposed, or likely-exposed, to a flavivirus, for example, by being bitten by a mosquito in an area where the mosquitos are known or suspected to be carriers of the flavivirus. In still other embodiments, the Z-medicaments are administered to a person who is in, or will be entering, an area where the mosquitos are known or suspected to be carriers of the flavivirus. In aspects of these embodiments, particular flavivirus groups or individual viruses, for example as described above, are specifically included or excluded. In other aspects of these embodiments, particular compounds or compositions of the Z-medicaments are specifically included or excluded.

The term “treating” or “treatment” broadly includes any kind of treatment activity, including the diagnosis, mitigation, or prevention of disease in humans or animals, or any activity that otherwise affects the structure or any function of the body. Treatment activity includes the administration of the medicaments, dosage forms, and pharmaceutical compositions described herein to a patient, especially according to the various methods of treatment disclosed herein, whether by a healthcare professional, the patient him/herself, or any other person. Treatment activities include the orders, instructions, and advice of healthcare professionals such as physicians, physician's assistants, nurse practitioners, and the like, that are then acted upon by any other person including other healthcare professionals or the patient him/herself. In some embodiments, treatment activity can also include encouraging, inducing, or mandating that a particular medicament, or combination thereof, be chosen for treatment of a condition—and the medicament is actually used—by approving insurance coverage for the medicament, denying coverage for an alternative medicament, including the medicament on, or excluding an alternative medicament, from a drug formulary, or offering a financial incentive to use the medicament, as might be done by an insurance company or a pharmacy benefits management company, and the like. In some embodiments, treatment activity can also include encouraging, inducing, or mandating that a particular medicament be chosen for treatment of a condition—and the medicament is actually used—by a policy or practice standard as might be established by a hospital, clinic, health maintenance organization, medical practice or physicians group, and the like. All such orders, instructions, and advice are to be seen as conditioning receipt of the benefit of the treatment on compliance with the instruction. In some instances, a financial benefit is also received by the patient for compliance with such orders, instructions, and advice. In some instances, a financial benefit is also received by the healthcare professional for compliance with such orders, instructions, and advice.

A pharmaceutical composition is one intended and suitable for the treatment of disease in humans. That is, it provides overall beneficial effect and does not contain amounts of ingredients or contaminants that cause toxic or other undesirable effects unrelated to the provision of the beneficial effect. A pharmaceutical composition will contain one or more active agents and may further contain solvents, buffers, diluents, carriers, and other excipients to aid the administration, solubility, absorption or bioavailability, and or stability, etc. of the active agent(s) or overall composition.

A compound or a composition disclosed herein, that is, the Z-medicaments, can be administered using a variety of routes. Routes of administration suitable for treating a flavivirus infection as disclosed herein generally provide systemic delivery. In various embodiments, the route of administration can be oral or by subcutaneous or intravenous injection. As currently approved, bortezomib is intended for subcutaneous or intravenous injection.

Aspects of the present specification provide, in part, administering a therapeutically or prophylactically effective amount of a compound or a composition disclosed herein. As used herein, the term “therapeutically effective amount” is synonymous with “therapeutically effective dose” and when used in reference to treating a flavivirus infection means at least the minimum dose of a compound or composition disclosed herein necessary to achieve the desired therapeutic or prophylactic effect. In some embodiments, it refers to an amount sufficient to prevent or disrupt the infection process, or to reduce the extent or duration of infection. In some embodiments, it includes a dose sufficient to reduce a symptom associated with the flavivirus infection. An effective dosage amount of a compound or a composition disclosed herein can readily be determined by the person of ordinary skill in the art considering all criteria (for example, the rate of excretion of the compound or composition used, the pharmacodynamics of the compound or composition used, the nature of the other compounds to be included in the composition, the particular route of administration, the particular characteristics, history and risk factors of the individual, such as, e.g., age, weight, general health and the like, the response of the individual to the treatment, or any combination thereof) and utilizing his best judgment on the individual's behalf.

### EXAMPLES

The following non-limiting examples are provided for illustrative purposes only in order to facilitate a more complete understanding of representative embodiments now contemplated. These examples should not be construed to limit any of the embodiments described in the present specification,

### Example 1

### Identification of Lead Compounds with Broad-Spectrum Activity Against Multiple ZIKV Strains

Materials and Methods

Cells and Viruses.

Vero E6 and LLC-MK2 cells were maintained in Dulbecco's Modified Eagle Medium (DMEM) supplemented with 8% fetal bovine serum (FBS) and penicillin and streptomycin (P/S). C6/36 cells were maintained in Eagle's Minimal Essential Medium (EMEM) supplemented with 5% FBS and P/S. ZIKV strains, including human strains PAN2016 (2016/Panama), R116265 (2016/Mexico), PAN2015 (2015/Panama), FLR (2015/Colombia), R103451 (2015/Honduras), PRVABC59 (2015/Puerto Rico), PLCal_ZV (2013/Thailand), and IbH 30656 (1968/Nigeria), mosquito strain MEX 2-81 (2016/Mexico), and rhesus macaque strain MR 766 (1947/Uganda), were used in the studies. These ZIKV strains were cultured in Vero E6 cells, and viral titers were detected by a standard plaque-forming assay. DENV human strains, including type 1: DENV-1-V1792 (2007/Vietnam), type 2: DENV-2-V594 (2006/Puerto Rico), type 3: DENV-3-V1043 (2006/Puerto Rico), and type 4: DENV-4-PR 06-65-740 (2006/Puerto Rico), were cultured in C6/36 cells, and the viral titers were determined by plaque-forming assay as described above.

Detection of Antiviral Activity of Compounds Against ZIKV Infections.

A plaque reduction inhibition assay was carried out to measure the inhibitory activity of Z-medicaments against infections of ZIKV and DENV. Briefly, ZIKV (strain PAN2016, 70-100 plaque-forming unit (PFU)) was incubated with 2-fold serial dilutions of Z-medicaments (including curcumin, digitonin, and conessine, as well as anti-ZIKV compound control, bortezomib) or DMSO (0.4% vol/vol) control at 37° C. for 1 h. The compound-virus mixtures were then transferred to Vero E6 cells (10⁵/well) and incubated at 37° C. for 1 h. For gossypol, ZIKV (strain PAN2016, ˜2.5×10³ PFU) was incubated with serial dilutions of this Z-medicament at 37° C. for 1 h, and the unbound gossypol was removed by centrifugation after addition of 3% PEG-6000. Gossypol-treated ZIKV was then incubated with Vero E6 cells at 37° C. for 1 h. The cells were washed thoroughly with PBS, and overlaid with DMEM containing 1% carboxymethyl cellulose and 2% FBS, followed by in vitro culture at 37° C. for 4-5 days and then staining with 0.5% crystal violet. The 50% inhibitory concentration (IC₅₀) of the Z-medicaments was calculated based on the dilutions at 50% plaque reduction using the CalcuSyn program.

Detection of In Vitro Cytotoxicity of the Disclosed Compounds in Vero E6 Cells.

The cytotoxicity of the Z-medicaments in Vero E6 (ZIKV target cells) was evaluated using the Cell Counting Kit-8 (CCK8). Briefly, 2-fold serial dilutions of Z-medicaments (100 μl/well) were added to equal volumes of cells (2.0×10⁴/well) in 96-well plates, and cultured at 37° C. for 3 days. The cells were then incubated with CCK8 solution, and absorbance measured at 450 nm (A450 value) using microplate reader. The 50% cytotoxic concentration (CC₅₀) of the Z-medicaments was calculated based on the percent cytotoxicity.

Results

Identification of Lead Compounds with Broad-Spectrum Activity Against Multiple ZIKV Strains

Using a plaque-based assay, we initially screened 720 compounds from a library for their inhibitory activity against infection of a recent ZIKV human strain (PAN2016). Under 20 μM of concentration, four “hit” Z-medicaments, including gossypol, curcumin, digitonin, and conessine (FIG. 1), demonstrated their inhibitory activity against ZIKV infection in Vero E6 cells at different levels. Among them, curcumin has been previously reported to inhibit ZIKV infection, whereas the other three Z-medicaments have not been previously reported to have anti-ZIKV activity. Gossypol, curcumin, digitonin, and conessine had ≥95% purity, with the IC₅₀ values of 3.48, 13.67, 4.31, and 9.75 μM, respectively, against ZIKV (strain PAN2016) (Table 1). The cytotoxicity of these four compounds was detected by a cell-based cytotoxicity assay in Vero E6 cells, and their CC₅₀ values ranged from 14.17 to 323.71 μM.

The identified Z-medicaments were further studied for their broad-spectrum activity against nine additional ZIKV strains, including those isolated from different hosts, namely humans, mosquitos, and rhesus macaques, at different time periods (1947-2016) in different countries, including Mexico, Panama, Columbia, Honduras, Puerto Rico, Thailand, Nigeria, and Uganda. The results showed that these Z-medicaments inhibit infection by all nine ZIKV strains tested with various IC₅₀ values (Table 1). Particularly, gossypol exhibited the most potent inhibitory activity with the IC₅₀ values ranging from 0.21 to 4.31 μM against all nine ZIKV strains tested (Table 1). It is also more potent than bortezomib, the previously reported anti-ZIKV compound as an active compound control, against all ZIKV strains tested (Table 1).

TABLE 1
Inhibitory activity of compounds against infections of ZIKV with different strains

| | | IC₅₀ (μM) against ZIKV strains | | | | |
|---|---|---|---|---|---|---|
| Z-medicament | CC₅₀ (μM) | PAN2016 | R116265 | PAN2015 | FLR | R103451 |
| Gossypol | 14.17 ± 0.74 | 3.48 ± 0.03 | 4.20 ± 0.08 | 3.95 ± 0.05 | 0.21 ± 0.01 | 2.28 ± 0.10 |
| Curcumin | 52.86 ± 0.52 | 13.67 ± 0.72 | 14.04 ± 0.15 | 13.71 ± 0.37 | 16.57 ± 0.34 | 11.22 ± 0.37 |
| Digitonin | 56.29 ± 1.20 | 4.31 ± 0.23 | 6.52 ± 0.59 | 5.00 ± 0.01 | 3.34 ± 0.22 | 4.30 ± 0.43 |
| Conessine | 323.71 ± 0.25 | 9.75 ± 0.26 | 7.18 ± 0.13 | 7.98 ± 0.29 | 9.65 ± 0.58 | 11.60 ± 0.33 |
| Bortezomib | 16.96 ± 0.20 | 9.75 ± 0.03 | 8.94 ± 0.10 | 9.88 ± 0.12 | 9.62 ± 0.59 | 14.14 ± 0.85 |
| | | IC₅₀ (μM) against ZIKV strains | | | | |
| Z-medicament | CC₅₀ (μM) | PRVABC59 | PLCal_ZV | IbH 30656 | MEX 2-81 | MR 766 |
| Gossypol | 14.17 ± 0.74 | 4.31 ± 0.02 | 1.98 ± 0.07 | 3.31 ± 0.11 | 2.79 ± 0.01 | 3.75 ± 0.01 |
| Curcumin | 52.86 ± 0.52 | 12.85 ± 0.35 | 10.84 ± 0.73 | 13.63 ± 0.31 | 5.62 ± 0.52 | 11.42 ± 0.29 |
| Digitonin | 56.29 ± 1.20 | 3.76 ± 1.12 | 3.19 ± 0.25 | 5.30 ± 0.13 | 3.84 ± 0.12 | 3.77 ± 0.31 |
| Conessine | 323.71 ± 0.25 | 9.08 ± 0.33 | 8.11 ± 0.37 | 10.25 ± 0.41 | 10.94 ± 0.06 | 7.44 ± 0.11 |
| Bortezomib | 16.96 ± 0.20 | 11.72 ± 0.82 | 31.04 ± 0.71 | 9.35 ± 0.23 | 7.67 ± 0.31 | 9.51 ± 0.26 |

Note:
The experiments were performed on Vero E6 cells, and the cytotoxicity of the Z-medicaments in this cell line is expressed as 50% cytotoxic concentration (CC₅₀). The inhibitory activity of the Z-medicaments against ZIKV infection is expressed as 50% inhibitory concentration (IC₅₀). Bortezomib was used as an anti-ZIKV compound control. The data are expressed as mean ± standard error of the mean (s.e.m.) (n = 2). The experiments were repeated twice with similar results.

Comparison of ZIKV E and NS2B-NS3 protein sequences revealed that most of the amino acid sequences were highly conserved, but that some variations among the ten ZIKV strains used for evaluation of the inhibitory activity of the Z-medicaments occurred, including the PAN2016 strain tested earlier. The above data demonstrate that the identified Z-medicaments, particularly gossypol, were able to block infections of divergent human, mosquito, and monkey ZIKV strains isolated from different time periods and countries, including six recent ZIKV human strains, confirming their broad-spectrum anti-ZIKV activity.

### Example 2

### Identification of Inhibition Mechanisms of Lead Compounds Against ZIKV Infection

Materials and Methods

Time-of-addition experiments. were performed to identify mechanisms of the Z-medicaments against ZIKV infection. Briefly, Vero E6 cells (10⁵/well) and/or ZIKV were incubated, at different infection steps as described below with or without the tested Z-medicaments at the specified concentrations of 15 μM for gossypol, 30 μM for curcumin, 7.5 μM for digitonin, 30 μM for conessine, or 10 μM for anti-ZIKV compound control bortezomib, for 1 h before ZIKV infection, 1 h after infection, or the same time during infection. After culture of the ZIKV- and/or compound-treated cells at 37° C. for 4-5 days, plaques were visualized with crystal violet staining, as described above, and the percent inhibition of the Z-medicaments was calculated. Specifically, the following six stages of ZIKV infection were tested: (a) Pretreatment of ZIKV (PAN2016, ˜2.5×10³ PFU) with the Z-medicaments at 37° C. for 1 h before incubation with cells; (b) Pretreatment of cells with the Z-medicaments at 37° C. for 1 h before incubation with ZIKV (PAN2016, ˜100 PFU); (c) Co-treatment of cells, ZIKV (PAN2016, ˜300 PFU) and the Z-medicaments at 4° C. for 1 h; (d) Co-treatment of cells, ZIKV (PAN2016, ˜100 PFU) and the Z-medicaments at 37° C. for 1 h; (e) Pre-incubation of cells with ZIKV (PAN2016, ˜300 PFU) at 4° C. for 1 h and then incubation with the Z-medicaments at 37° C. for 1 h; and (f) Pre-incubation of ZIKV (PAN2016, ˜100 PFU) and cells at 37° C. for 1 h, followed by incubation with the Z-medicaments at 37° C. for 1 h.

Results

Identification of Inhibition Mechanisms of Lead Compounds, Including Gossypol, Against ZIKV Infection

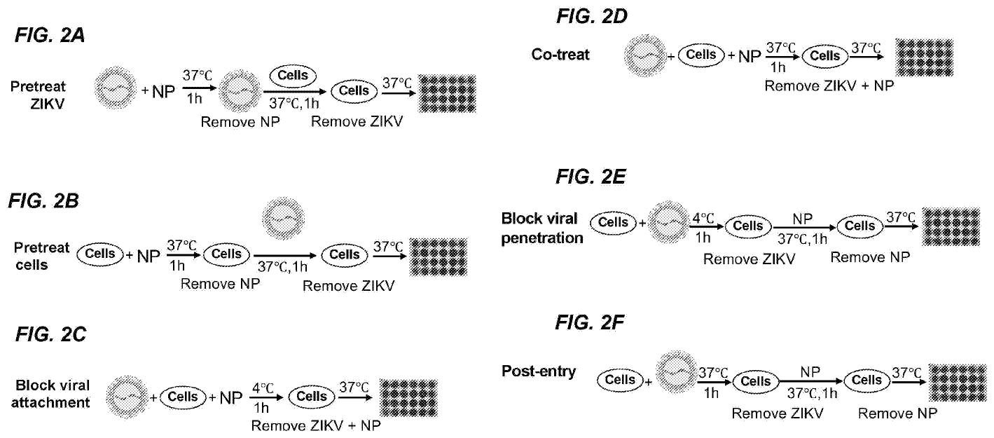

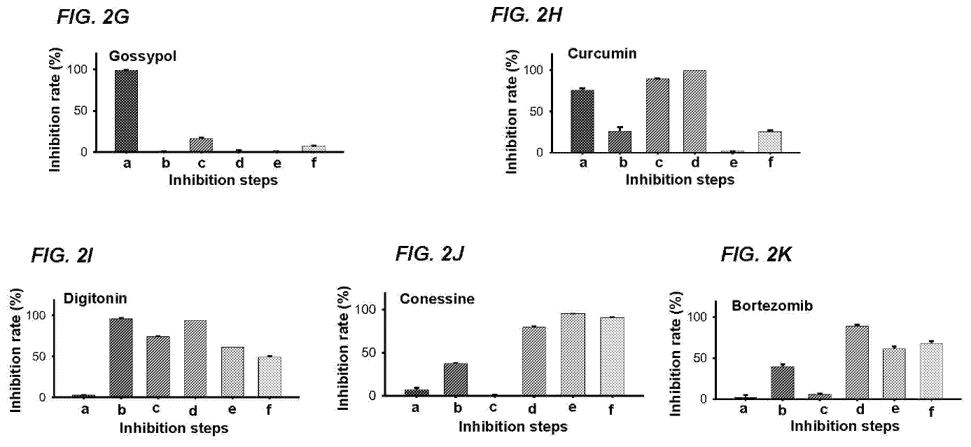

To identify which step(s) of ZIKV infection in its life cycle were blocked by these compounds, we carried out a time-of-addition experiment by incubation of the compounds with ZIKV and/or cells at different time points during ZIKV and cell interaction, and then calculated the percent inhibition based on the number of plaques formed. To test whether a Z-medicament can neutralize ZIKV infection or inhibit viral entry by targeting the viral proteins, ZIKV was pre-treated with the Z-medicament at 37° C. before incubation with the host cells (FIG. 2A). To evaluate whether a Z-medicament can bind to the cellular receptor(s) or co-factor(s) to block virus-receptor binding, cells were pre-treated with the Z-medicament at 37° C. before incubation with ZIKV (FIG. 2B). To determine whether a Z-medicament can inhibit attachment of ZIKV to target cells (but not blocking the virus-cell membrane fusion), cells were co-treated with ZIKV at 4° C. in the presence of the Z-medicament (FIG. 2C). To assess whether a Z-medicament can inhibit attachment of ZIKV to target cells and subsequent virus-cell membrane fusion, the cells were co-treated with ZIKV and the Z-medicament at 37° C. (FIG. 2D). To investigate whether a Z-medicament can inhibit ZIKV fusion with the cell membrane and then entry into the cell, cells were pre-treated with ZIKV at 4° C. first and then incubated with the Z-medicament at 37° C. (FIG. 2E). To study whether a Z-medicament can inhibit ZIKV infection at post-entry stages, i.e., viral replication, virion assembly, or release, cells were pre-treated with ZIKV and then incubated with the Z-medicament at 37° C. (FIG. 2F).

These experiments provided insight into the potential mechanisms of the Z-medicaments responsible for inhibiting ZIKV (PAN2016) infection. After pretreatment of ZIKV with gossypol at 37° C. before incubation with the target cells, ZIKV completely lost its infectivity, whereas it maintained its infectivity after other treatments described above (FIG. 2G), suggesting that gossypol can effectively neutralize ZIKV infection by targeting the virus, rather than the cell or cell-associated entry or replication stages.

The results from curcumin revealed that about 75-100% of ZIKV infection was blocked when curcumin was incubated with ZIKV only at 37° C., or co-incubated with ZIKV and cells at 4° C. or 37° C., whereas there was low to no impact on ZIKV infection when curcumin was pre-treated with cells, or post-incubated with ZIKV-treated cells at 4° C. and 37° C., respectively (FIG. 2H). These results suggest that curcumin inhibits ZIKV infection at the early stage(s) of viral entry, particularly viral attachment stage.

Pretreatment of Vero E6 cells with digitonin and then with ZIKV, or co-treatment of Vero E6 cells with ZIKV and digitonin, at 37° C. significantly (≥94%) blocked ZIKV infection, whereas pre-incubation of cells with ZIKV and then with digitonin at 37° C., pre-incubation of cells with ZIKV at 4° C. and then with digitonin at 37° C., or incubation of cells with ZIKV and digitonin at 4° C., inhibited about 49-74% of ZIKV infection (FIG. 2I). In contrast, pre-incubation of digitonin and ZIKV had no effects on ZIKV infection. These results suggest that digitonin could not directly neutralize ZIKV infection, but inhibited ZIKV infection by binding to the viral receptor(s) or inhibiting viral entry, i.e., attachment and membrane fusion, and/or post-entry steps.

The data from conessine indicated that co-incubation of cells with conessine and ZIKV at 37° C., or post-incubation of conessine with ZIKV-treated cells at 4° C. or 37° C. resulted in 80-96% inhibition of ZIKV infection, whereas pretreatment of cells with conessine before ZIKV incubation blocked about 38% of ZIKV infection. Nevertheless, pre-incubation of conessine and ZIKV at 37° C., or co-treatment of cells with conessine and ZIKV at 4° C., had very low, to no, effects on ZIKV infection (FIG. 2J). These data suggest that conessine does not block ZIKV attachment to the host cell, but inhibits ZIKV infection by targeting virus-cell fusion or a post-entry step, the mechanism similar to that of the control anti-ZIKV compound bortezomib (FIG. 2K).

Therefore, the above data confirm the potent inhibitory activity of the identified Z-medicaments, particularly gossypol, in blocking ZIKV infection at various stages of the viral life cycle.

### Example 3

### Detection of Binding Region(s) or Site(s) of Lead Compounds in ZIKV Proteins

Materials and Methods

Construction and Expression of ZIKV NS2B-NS3 Protease

Recombinant ZIKV NS2B-NS3 protease was constructed and expressed in an E. coli expression system. Briefly, the genes encoding NS2B protein (residues 49-97) of ZIKV (GenBank accession no. NC_012532) was fused with NS3 protein (residues 1-185) through a covalent linker (Gly4-Thr-Gly4), which were then cloned into the pET-28b(+) expression vector with a C-terminal Hiss tag. The ZIKV NS2B-NS3 protein was expressed in inclusion bodies of E. coli after addition of isopropyl-VD-thiogalactopyranoside (IPTG, final concentration 1 mM) and culture at 28° C. for 12 h, followed by purification using Ni-NTA affinity chromatography.

ELISA.

The binding between the Z-medicament and ZIKV full-length E protein, EDIII protein (E residues 298-409 fused with a C-terminal human Fc), or NS2B-NS3 protease was carried out by ELISA. Briefly, ELISA plates were pre-coated with the proteins described above at 4° C. overnight, and blocked with 2% fat-free milk at 37° C. for 2 h. Serial dilutions of the Z-medicament or DMSO (control) were then added to the plates and incubated at 37° C. for 2 h. The plates were washed with PBS containing Tween-20 (PBST), and incubated at 37° C. for 1 h with ZIKV EDIII-specific human mAb ZKA64-LALA (0.5 μg/ml) (for binding to ZIKV full-length E and EDIII proteins), or mouse sera specific to ZIKV NS2B-NS3 (for binding to NS2B-NS3 protease). The plates were washed with PBST, and incubated with horseradish peroxidase (HRP)-conjugated anti-human IgG-Fab (1:3000) or anti-mouse IgG (1:3000) antibody at 37° C. for 1 h. The 3,3′,5,5′-tetramethylbenzidine (TMB) substrate was added to the plates, and the reaction was stopped by 1 N H₂SO₄. Absorbance at 450 nm (A450 value) was measured by ELISA microplate reader. EC₅₀ (50% effective concentration) was calculated as described above.

To determine the ability of gossypol to inhibit binding between ZIKV EDIII protein and EDIII-specific human mAbs (SMZAb5, ZKA64-LALA, ZV-67, or Z004), ELISA was carried out, as described above, except that serially diluted gossypol or DMSO (control) was added in the presence of mAbs (0.5 μg/ml), followed by sequential incubation with HRP-conjugated anti-human IgG-Fab antibody and TMB substrate, and detection to yield a A450 value. The percent inhibition of the Z-medicaments was calculated, and IC₅₀ (concentration representing 50% reduction in EDIII-mAb binding) was obtained using the CalcuSyn program, as described above.

Surface Plasmon Resonance (SPR).

The interactions between the Z-medicaments and ZIKV full-length E or NS2B-NS3 protease were analyzed at 25° C. using the Biacore T200 system. Briefly, ZIKV E or NS2B-NS3 protein was immobilized on a sensor chip (CM5) using the Amine Coupling Kit. The disclosed Z-medicaments at various concentrations were subsequently injected as analytes, and PBS-P (20 mM phosphate buffer containing 2.7 mM KCl, 137 mM NaCl, and 0.05% surfactant P20, pH 7.4) was used as the running buffer. The data were analyzed using Biacore evaluation software (T200 version 1.0), and the curve was fitted with a 1:1 binding model.

Inhibition of NS2B-NS3 Protease Activity.

The inhibition of the activity of NS2B-NS3 protease by the disclosed Z-medicaments was carried out as follows. Briefly, a fluorescence-based enzymatic assay was conducted to detect the activity of NS2B-NS3 protease using benzoyl-norleucinelysine-lysine-arginine 7-amino-4-methylcoumarine (Bz-Nle-Lys-Lys-Arg-AMC) as a substrate. The fluorescence signal released from AMC was measured at 460 nm with excitation at 355 nm, using a microplate reader. Serial dilutions of compounds or DMSO (control) were incubated with ZIKV NS2B-NS3 protein (1 μg/ml) at 37° C. for 1 h, followed by addition of the aforementioned substrate (4 μM) to initiate the cleavage to detect whether the Z-medicaments inhibited protease activity. After 10 min, the fluorescence intensity was measured as noted above. The reaction and dilution buffers contained 10 mM Tris-HCl, 20% glycerol, 1 mM CHAPS, and 5% DMSO, pH 8.5. The percent inhibition by the Z-medicaments was calculated, and IC₅₀ (concentration causing 50% reduction in protease activity) was obtained, as described above.

Results

Identification of Binding Region(s) or Site(s) of Lead Compounds in ZIKV Proteins

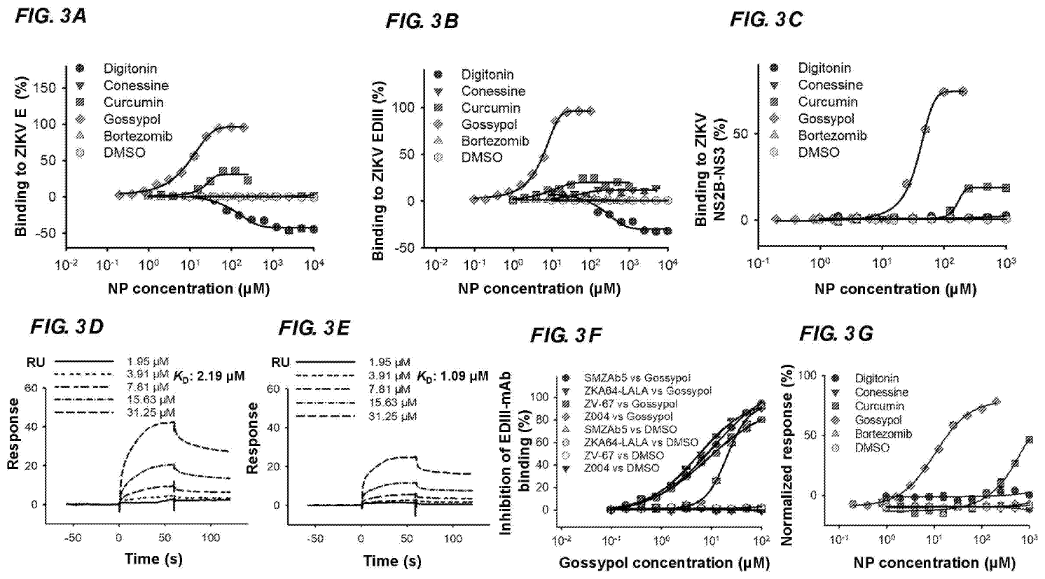

To identify the binding region(s) of the Z-medicaments in ZIKV proteins, we first carried out an ELISA-based approach by coating the plates with ZIKV full-length E (FIG. 3A), EDIII (FIG. 3B), or NS2B-NS3 (FIG. 3C) proteins. We then tested for the binding affinity using a ZIKV EDIII-specific mAb, ZKA64-LALA for E or EDIII binding, or NS2B-NS3 protein-immunized mouse sera for NS2B-NS3 binding. Results revealed that gossypol bound potently to all three proteins tested, with EC₅₀ values of 7.12, 4.22, and 56.09 μM, respectively, for full-length E, EDIII, and NS2B-NS3 proteins, whereas curcumin had much lower binding affinity to ZIKV full-length E and NS2B-NS3 proteins. Otherwise, digitonin, conessine, bortezomib (anti-ZIKV compound control), and DMSO (negative control), bound to no ZIKV proteins tested. We then evaluated the binding of gossypol using an SPR assay, and the results showed that it had binding affinity values of 2.19 and 1.09 μM, respectively, against ZIKV E and NS2B-NS3 proteins (FIG. 3D-E).

Since gossypol bound to ZIKV E protein, particularly the EDIII region, we further carried out an ELISA completion assay to identify its potential binding site(s) in the EDIII. Accordingly, ZIKV EDIII protein was coated on the plates, and the binding between EDIII and EDIII-specific mAbs (SMZAb5, ZKA64-LALA, ZV-67, or Z004) was evaluated in the presence of serially diluted gossypol. The results showed that gossypol potently blocked the EDIII-mAb binding in a dose-dependent manner, with the IC₅₀ values of 7.32, 5.72, 11.7, and 22.2 μM, respectively, against SMZAb5, ZKA64-LALA, ZV-67, or Z004 mAbs, whereas DMSO control showed no blockage of this binding (FIG. 3F). These mAbs have potent neutralizing activity against ZIKV infection, and recognize epitopes, including the lateral ridge, such as residues 309-314, 331-337, 368, 370, 371, and 393-397, of ZIKV EDIII protein. Therefore, the above data suggest that gossypol most likely binds to the lateral ridge of the ZIKV EDIII protein to block the EDIII-mAb binding.

As described earlier, in addition to binding to ZIKV EDIII, gossypol also bound to ZIKV NS2B-NS3 protease. To evaluate the ability of gossypol to block the cleavage activity of ZIKV NS2B-NS3 protease, we carried out a fluorescence-based inhibition assay in the presence of serially diluted gossypol, and measured the subsequent fluorescence signals. Results indicated that gossypol inhibited the cleavage of ZIKV NS2B-NS3 protease in a dose-dependent manner, with an IC₅₀ value of 28.52 μM. In contrast, curcumin had low inhibitory activity, whereas other Z-medicaments and DMSO control had no activity at all to inhibit this protease activity (FIG. 3G), reflecting their low to no binding to NS2B-NS3 protease. These results confirmed the ability of gossypol to strongly inhibit ZIKV NS2B-NS3 protease activity. NS2B-NS3 protease is essential for post-entry/post-translational polyprotein processing in viral life cycle

The above data demonstrate that gossypol bound strongly to ZIKV EDIII and conserved NS2B-NS3 protease, thus blocking the EDIII-mAb binding at important neutralizing epitopes and inhibiting NS2B-NS3 protease activity. These data reasonably explain the potent broad-spectrum antiviral activity of gossypol against infections of multiple ZIKV strains.

### Example 4

### Synergistic Effects of Gossypol with Other Z-Medicaments Against ZIKV Infection

Materials and Methods

Combinatorial Effects of Gossypol with Other Z-Medicaments Against ZIKV Infection.

The potential synergistic effect of gossypol with other Z-medicaments was carried out as follows. Briefly, ZIKV (strain PAN2016, FLR, or PRVABC59, 2.5×10³-PFU) was incubated with serially diluted gossypol at 37° C. for 1 h, and the unbound gossypol was removed by centrifugation after addition of 3% PEG-6000. The gossypol-treated ZIKV was incubated with Vero E6 cells at 37° C. for 1 h in the presence of DMEM containing serial dilutions of another Z-medicament, such as curcumin, digitonin, conessine, or bortezomib. The unbound viruses and Z-medicaments were removed, and the cells were cultured at 37° C. for 4-5 days, followed by staining with 0.5% crystal violet. The Z-medicaments without combinations were used as controls. The IC₅₀ of the Z-medicaments was calculated as described above.

The Z-medicaments were then analyzed for synergistic effects based on the combination index (CI) and IC₅₀ values using the CalcuSyn program. Specifically, CI values <1 and >1 indicate synergy and antagonism, respectively. Synergy was further identified as five different categories: CI values <0.1, 0.1-0.3, 0.3-0.7, 0.7-0.85, and 0.85-0.90 indicate very strong synergism, strong synergism, synergism, moderate synergism, and slight synergism, respectively. Fold enhancement of anti-ZIKV potency is expressed as the ratio of molar concentrations of the Z-medicaments tested alone and in the mixture.

Results

Gossypol has Significant Synergistic Effects with Other Z-Medicaments Against ZIKV Infection

Since gossypol demonstrated the highest antiviral activity individually against almost all ZIKV strains tested, we next investigated the potential synergistic effects of the combination of gossypol with the three other Z-medicaments identified, namely curcumin, digitonin, and conessine, as well as anti-ZIKV compound control (bortezomib). Results demonstrated that there were significant synergistic inhibitory effects against three ZIKV strains (PAN2016, FLR, and PRVABC59) tested when combining gossypol with any of these Z-medicaments, and the CI values ranged from 0.44 to 0.6 μM, from 0.44 to 0.95 μM, and from 0.19 to 0.3 μM, respectively, for ZIKV PAN2016, FLR, and PRVABC59 strains, respectively (Tables 2-4). The combinations of gossypol with each of these Z-medicaments also resulted in the highest-fold enhancement of anti-PRVABC59 activity among the three ZIKV strains tested (Table 4). These data show that gossypol can be combined with other inhibitors described above to further increase overall inhibitory activity against current and future emergent ZIKV strains.

### Example 5

### Potent Inhibitory Activity of Lead Compounds, Particularly Gossypol, Against Infections of Four Serotypes of Dengue Virus (DENV-1-4)

Materials and Methods

Detection of In Vitro Cytotoxicity of Z-Medicaments in LLC-MK2 Cells.

The cytotoxicity of Z-medicaments in LLC-MK2 (DENV-1-4 target cells) was detected using Cell Counting Kit-8 (CCK8). Briefly, 2-fold serial dilutions of Z-medicament (100 μl/well) was added to equal volumes of cells (2.0×10⁴/well) in 96-well plates, and cultured at 37° C. for 3 days. The cells were then incubated with CCK8 solution, and measured absorbance at 450 nm (A₄₅₀ value) using a microplate reader. The 50% cytotoxic concentration (CC₅₀) of the Z-medicaments was calculated based on the percent cytotoxicity using the CalcuSyn program.

Detection of Antiviral Activity of Z-Medicaments Against DENV-1-4 Infections.

The inhibitory activity of the Z-medicaments against DENV-1-4 was performed following the similar procedures as for ZIKV, except that LLC-MK2 cells were used for the infection, and cells were cultured at 37° C. for 14-16 days before staining with 0.5% crystal violet. The 50% inhibitory concentration (IC₅₀) of the Z-medicaments was calculated based on the dilutions at 50% plaque reduction using the CalcuSyn program, as described above.

TABLE 2
Combinatorial effects of gossypol with other compounds in
inhibition of infection of ZIKV PAN2016 strain

| | IC₅₀ (μM) | | Fold of | |
|---|---|---|---|---|
| Z-medicament | Alone | In Mixture | Enhancement | CI |
| Gossypol | 3.79 ± 0.01 | 0.93 ± 0.04 | 4.08 | |
| | 3.79 ± 0.01 | 1.08 ± 0.19 | 3.51 | |
| | 3.79 ± 0.01 | 0.81 ± 0.11 | 4.68 | |
| | 3.79 ± 0.01 | 1.00 ± 0.02 | 3.79 | |
| Curcumin | 13.20 ± 0.81 | 3.67 ± 0.18 | 3.60 | 0.52 |
| Digitonin | 4.85 ± 0.24 | 1.51 ± 0.27 | 3.21 | 0.60 |
| Conessine | 10.04 ± 0.25 | 2.26 ± 0.30 | 4.44 | 0.44 |
| Bortezomib | 10.65 ± 0.01 | 2.79 ± 0.06 | 3.82 | 0.53 |

Note:
The experiments were performed on Vero E6 cells, and the inhibitory of the Z-medicaments against infection of ZIKV (strain PAN2016) is expressed as 50% inhibitory concentration (IC₅₀). Ratios of molar concentrations of gossypol and curcumin, digitonin, conessine (three lead compounds), of bortezomib (anti-ZIKV compound control) in combination against ZIKV strain PAN2016 (2.5 × 10³ − PFU) are 0.29:1, 0.78:1, 0.38:1, and 0.36:1, respectively. The data expressed as mean ± s.e.m. (n = 2). The experiments were repeated twice with similar results.
CI, combination index.

TABLE 3
Combinatorial effects of gossypol with other compounds in
inhibition of infection of ZIKV FLR strain

| | IC₅₀ (μM) | | Fold of | |
|---|---|---|---|---|
| Z-medicament | Alone | In Mixture | Enhancement | CI |
| Gossypol | 0.26 ± 0.01 | 0.06 ± 0.01 | 4.33 | |
| | 0.26 ± 0.01 | 0.12 ± 0.01 | 2.17 | |
| | 0.26 ± 0.01 | 0.10 ± 0.01 | 2.60 | |
| | 0.26 ± 0.01 | 0.05 ± 0.01 | 5.20 | |
| Curcumin | 17.05 ± 0.08 | 4.44 ± 0.74 | 3.84 | 0.49 |
| Digitonin | 3.86 ± 0.02 | 1.89 ± 0.13 | 2.04 | 0.95 |
| Conessine | 10.07 ± 0.45 | 4.73 ± 0.08 | 2.13 | 0.85 |
| Bortezomib | 9.70 ± 0.76 | 2.40 ± 0.28 | 4.04 | 0.4 |

Note:
The experiments were performed on Vero E6 cells, and the inhibitory activity of the Z-medicaments against infection of ZIKV (strain FLR) is expressed as 50% inhibitory concentration (IC₅₀). Ratios of molar concentrations of gossypol and curcumin, digitonin, conessine (three lead compounds), or bortezomib (anti-ZIKV compound control) in combination against ZIKV strain FLR (2.5 × 10³ − PFU) are 0.02:1, 0.07:1, 0.03:1, and 0.03:1, respectively. The data are expressed as mean ± s.e.m. (n = 2). The experiments were repeated twice with similar results.

TABLE 4
Combinatorial effects of gossypol with other compounds in
inhibition of infection of ZIKV PRVABC59 strain

| | IC₅₀ (μM) | | Fold of | |
|---|---|---|---|---|
| Z-medicament | Alone | In Mixture | Enhancement | CI |
| Gossypol | 4.38 ± 0.08 | 0.65 ± 0.06 | 6.74 | |
| | 4.38 ± 0.08 | 0.63 ± 0.10 | 6.95 | |
| | 4.38 ± 0.08 | 0.62 ± 0.01 | 7.06 | |
| | 4.38 ± 0.08 | 0.45 ± 0.02 | 9.73 | |
| Curcumin | 12.46 ± 0.05 | 1.93 ± 0.16 | 6.46 | 0.30 |
| Digitonin | 3.84 ± 0.81 | 0.55 ± 0.09 | 6.98 | 0.29 |
| Conessine | 9.40 ± 0.21 | 1.29 ± 0.01 | 7.29 | 0.28 |
| Bortezomib | 12.17 ± 0.07 | 1.07 ± 0.04 | 11.37 | 0.19 |

Note:
The experiments were preformed on Vero E6 cells, and the inhibitory activity of the Z-medicaments against infection of ZIKV (strain PRVABC59) is expressed as 50% inhibitory concentration (IC₅₀). Ratios of molar concentrations of gossypol and curcumin, digitonin, conessine (three lead compounds), or bortezomib (anti-ZIKV compound control) in combination against ZIKV strain PRVABC59 (2.5 × 10³ − PFU) are 0.35:1, 1.14:1, 0.47:1, and 0.36:1, respectively. The data are expressed as mean ± s.e.m. (n = 2). The experiments were repeated twice with similar results.

TABLE 5
Inhibitory activity of Z-medicaments against
infections of DENV-1-4 (serotypes 1-4 of DENV)

| | | IC₅₀ (μM) | | | |
|---|---|---|---|---|---|
| | | | | | DENV- |
| Z- | CC₅₀ | DENV-1- | DENV-2- | DENV-3- | 4-PR 06- |
| medicament | (μM) | V1792 | V594 | V1043 | 65-740 |
| Gossypol | 14.54 ± 0.59 | 1.87 ± 0.01 | 1.89 ± 0.21 | 3.70 ± 0.59 | 2.60 ± 0.12 |
| Curcumin | 59.42 ± 1.18 | 9.37 ± 0.47 | 3.07 ± 0.07 | 2.09 ± 0.12 | 4.83 ± 0.24 |
| Digitonin | 59.02 ± 0.33 | 5.21 ± 0.35 | 6.56 ± 0.21 | 4.07 ± 0.83 | 6.44 ± 0.34 |
| Conessine | 302.69 ± 13.40 | 7.09 ± 0.08 | 6.61 ± 0.60 | 7.41 ± 0.04 | 7.27 ± 0.31 |

Note:
The experiments were performed on LLC-MK2 cells, and the cytotoxicity of the Z-medicaments in this cell line is expressed as 50% cytotoxic concentration (CC₅₀). The inhibitory activity of Z-medicaments against infections of DENV-1-4 is expressed as 50% inhibitory concentration (IC₅₀). The data are expressed as mean ± s.e.m. (n = 4). The experiments were repeated twice with siilar results.

Results

The Identified Lead Compounds, Particularly Gossypol, had Potent Inhibitory Activity Against Infections of all Four DENV Serotypes

Identification of broad-spectrum anti-flavivirus inhibitors is crucial to treat infections caused by ZIKV and other flaviviruses, such as DENV. Hence, using a plaque assay, we evaluated the antiviral activity of Z-medicaments against infections of four serotypes of DENV human strains in LLC-MK2 cells. Even though four Z-medicaments could inhibit DENV-1-4 infections, results showed that gossypol had the highest potency against DENV-1, DENV-2, and DENV-4 infections, with the IC₅₀ values of 1.87, 1.89 and 2.6 μM, respectively (Table 5). Also, the anti-DENV-3 activity of gossypol (IC₅₀ value: 3.7 μM) was only slightly higher than that of curcumin (IC₅₀ value: 2.09 μM). The cytotoxicity of these compounds on LLC-MK2 cells was investigated by a cytotoxicity assay, with the CC₅₀ values ranging from 14.54 to 302.69 μM. The above data indicate the potent anti-DENV activity of the four Z-medicaments identified, particularly gossypol, against infections of four DENV human strains with no cytotoxicity.

As described earlier, gossypol targeted E, mainly EDIII, and NS2B-NS3 proteins, of ZIKV. Although a number of variations have been identified in the amino acid sequences of E and NS2B-NS3 proteins of ZIKV and DENV strains tested in this study, gossypol could still inhibit all ZIKV and DENV strains tested, suggesting that it potentially targeted the conserved sequences in ZIKV and DENV EDIII and/or NS2B-NS3 proteins. Our data further explain the potent, broad-spectrum activity of gossypol against infections of at least two flaviviruses, including ZIKV and DENV.

TABLE 6
Amino acid sequences of Zika virus (ZIKV) and dengue virus
(DENV) strains used in the studies

| | |
|---|---|
| SEQ ID NO: 1: | ZIKV (ZikaSPH2015 strain) full-length E protein |
| | (containing ZIKV full-length E protein): |
| | IRCIGVSNRDFVEGMSGGTWVDIVLEHGGCVTVMAQDKPTVDIELVTTTVSNMAEVRSYC |
| | YEASISDMASDSRCPTQGEAYLDKQSDTQYVCKRTLVDRGWGNGCGLFGKGSLVTCAKFA |
| | CSKKMTGKSIQPENLEYRIMLSVHGSQHSGMIVNDTGHETDENRAKVEITPNSPRAEATL |
| | GGFGSLGLDCEPRTGLDFSDLYYLTMNNKHWLVHKEWFHDIPLPWHAGADTGTPHWNNKE |
| | ALVEFKDAHAKRQTVVVLGSQEGAVHTALAGALEAEMDGAKGRLSSGHLKCRLKMDKLRL |
| | KGVSYSLCTAAFTFTKIPAETLHGTVTVEVQYAGTDGPCKVPAQMAVDMQTLTPVGRLIT |
| | ANPVITESTENSKMMLELDPPFGDSYIVIGVGEKKITHHWHRSGSTIGKAFEATVRGAKR |
| | MAVLGDTAWDFGSVGGALNSLGKGIHQIFGAAFKSLFGGMSWFSQILIGTLLMWLGLNTK |
| | NGSISLMCLALGGVLIFLSTAVSAD |
| SEQ ID NO: 2: | ZIKV (ZikaSPH2015 strain) E protein domain III (EDIII) |
| | (containing ZIKV E protein residues 298-409): |
| | LRLKGVSYSLCTAAFTFTKIPAETLHGTVTVEVQYAGTDGPCKVPAQMAVDMQTLTPVGR |
| | LITANPVITESTENSKMMLELDPPFGDSYIVIGVGEKKITHHWHRSGSTIGK |
| SEQ ID NO: 3: | ZIKV non-structure proteins NS2B and NS3 (747 amino acids: |
| | NS2B (residues 1-130) + NS3 (resideus 1-617)): |
| | SWPPSEVLTAVGLICALAGGFAKADIEMAGPMAAVGLLIVSYVVSGKSVDMYIERAGDIT |
| | WEKDAEVTGNSPRLDVALDESGDFSLVEEDGPPMREIILKVVLMAICGMNPIAIPFAAGA |
| | WYVYVKTGKRSGALWDVPAPKEVKKGETTDGVYRVMTRRLLGSTQVGVGVMQEGVFHTMW |
| | HVTKGAALRSGEGRLDPYWGDVKQDLVSYCGPWKLDAAWDGLSEVQLLAVPPGERARNIQ |
| | TLPGIFKTKDGDIGAVALDYPAGTSGSPILDKCGRVIGLYGNGVVIKNGSYVSAITQGKR |
| | EEETPVECFEPSMLKKKQLTVLDLHPGAGKTRRVLPEIVREAIKKRLRTVILAPTRVVAA |
| | EMEEALRGLPVRYMTTAVNVTHSGTEIVDLMCHATFTSRLLQPIRVPNYNLYIMDEAHFT |
| | DPSSIAARGYISTRVEMGEAAAIFMTATPPGTRDAFPDSNSPIMDTEVEVPERAWSSGFD |
| | WVTDHSGKTVWFVPSVRNGNEIAACLTKAGKRVIQLSRKTFETEFQKTKNQEWDFVITTD |
| | ISEMGANFKADRVIDSRRCLKPVILDGERVILAGPMPVTHASAAQRRGRIGRNPNKPGDE |
| | YMYGGGCAETDEGHAHWLEARMLLDNIYLQDGLIASLYRPEADKVAAIEGEFKLRTEQRK |
| | TFVELMKRGDLPVWLAYQVASAGITYTDRRWCFDGTTNNTIMEDSVPAEVWTKYGEKRVL |
| | KPRWMDARVCSDHAALKSFKEFAAGKR |
| SEQ ID NO: 4 | ZIKV non-structure protein NS2B-NS3 protease [containing NS2B |
| | protein (residues 49-97) and NS3 protein (residues 1-185)]: |
| | VDMYIERAGDITWEKDAEVTGNSPRLDVALDESGDFSLVEEDGPPMREISGALWDVPAPK |
| | EVKKGETTDGVYRVMTRRLLGSTQVGVGVMQEGVFHTMWHVTKGAALRSGEGRLDPYWGD |
| | VGQDLVSYCGPWKLDAAWDGLSEVQLLAVPPGERARNIQTLPGIFKTKDGDIGAVALDYP |
| | AGTSGSPILDKCGRVIGLYGNGVVIKNGSYVSAITQGKREEETPVECFEPSMLK |
| SEQ ID NO: 5 | ZIKV NS2B-NS3 protease [containing NS2B protein (reidues |
| | 49-97) and NS2 protein (residues 1-185) through a covalent |
| | linker (Gly4-Thr-Gly4; underlined)]: |
| | VDMYIERAGDITWEKDAEVTGNSPRLDVALDESGDFSLVEEDGPPMREIGGGGTGGGGSG |
| | ALWDVPAPKEVKKGETTDGVYRVMTRRLLGSTQVGVGVMQEGVFHTMWHVTKGAALRSGE |
| | GRLDPYWGDVKQDLVSYCGPWKLDAAWDGVSEVQLLAVPPGERARNIQTLPGIFKTKDGD |
| | IGAVALDYPAGTSGSPILDKCGRVIGLYGNGVVIKNGSYVSAITQGKREEETPVECFEPS |
| | MLK |

### Example 6

### Potent Inhibitory Activity of Gossypol Derivatives

Materials and Methods

Animals.

Adult male (3-4 or 7-8-week-old) and pregnant female (8-12-week-old, E12-14) Ifnar1^{-/-} mice were used in the study. The animal studies were performed in strict accordance with recommendations in the Guide for the Care and Use of Laboratory Animals of the National Institutes of Health. The animal protocols were approved by the Committee on the Ethics of Animal Experiments of New York Blood Center (Permit Numbers: 344.02 and 345.02).

Antiviral Activity of Gossypol Derivatives.

A series of compounds (gossypol or its derivatives) were purchased from Timtech, and detected by plaque assay for their inhibitory activity against infection of ZIKV and DENV. Briefly, ZIKV (2.5×10³ PFU) was incubated with gossypol derivatives or gossypol (as control) at 37° C. for 1 h. After removal of the unbound compounds by centrifugation after addition of 3% PEG-6000, ZIKV was incubated with Vero E6 cells at 37° C. for 1 h. The cells were then washed with PBS, overlaid with DMEM containing 1% carboxymethyl cellulose and 2% FBS, and cultured at 37° C. for 4-5 days, followed by staining with 0.5% crystal violet. The inhibitory activity of gossypol derivatives or gossypol against DENV-1-4 was assessed as described above, except that LLC-MK2 cells were used for infection, and cells were cultured at 37° C. for 14-16 days, and then stained with 0.5% crystal violet. 50% inhibitory concentration (IC₅₀) of compounds were calculated.

In Vitro Cytotoxicity of Gossypol Derivatives.

The cytotoxicity of gossypol derivatives was detected in Vero E6 (for ZIKV) or LLC-MK2 cells (for DENV-1-4) using CCK8 kit according to the manufacturer's instructions. Briefly, compounds at 2-fold serial dilutions were added to cells (2.0×10⁴/well) pre-seeded in 96-well plates. The cells were cultured at 37° C. for 3 days, and incubated with CCK8 solution, followed by measurement of absorbance at 450 nm (A450 value) using microplate reader. The 50% cytotoxic concentration (CC₅₀) of compounds was calculated.

Time-of-Addition Experiment.

This experiment was carried out to detect potential inhibitory mechanisms of gossypol and its derivative ST087010. Briefly, Vero E6 cells (10⁵/well) and/or ZIKV were incubated with or without the above compounds (15 μM) for 1 h before, 1 h after, or the same time during infection of ZIKV. The following six steps of ZIKV infection were tested: 1) Step 1: Pretreatment of ZIKV (PAN2016, 2.5×10³ PFU) with each compound at 37° C. for 1 h, and then incubation with cells. Step 2: Pre-treatment of cells with each compound at 37° C. for 1 h, and then incubation with ZIKV (PAN2016, 100 PFU). Step 3: Attachment. Co-treatment of cells with ZIKV (PAN2016, ˜300 PFU) and each compound at 4° C. for 1 h. Step 4: Co-treatment of cells, ZIKV (PAN2016, 100 PFU), and each compound at 37° C. for 1 h. Step 5: Fusion. Pre-incubation of cells with ZIKV (PAN2016, 300 PFU) at 4° C. for 1 h, and then incubation with each compound at 37° C. for 1 h. Step 6: Post-entry. Preincubation of ZIKV (PAN2016, 100 PFU) and cells at 37° C. for 1 h, and then incubation with each compound at 37° C. for 1 h. After culturing at 37° C. for 4-5 days, the cells were stained with crystal violet, and plaques were visualized, based on which percent inhibition (% inhibition) of the compounds was calculated.

ELISA.

ELISA was carried out to detect the binding between compounds (gossypol or its derivative ST087010) and ZIKV full-length E, EDIII containing a C-terminal human Fc tag, or NS2B-NS3 protein. Briefly, ELISA plates were coated with each protein (1 μg/ml) at 4° C. overnight, and were then incubated with blocking buffer containing 2% fat-free milk in PBST at 37° C. for 2 h. The above compounds, or DMSO control, at serial dilutions were added to the plates, and incubated at 37° C. for 2 h. After three washes using PBST, the plates were incubated at 37° C. for 2 h with ZIKV EDIII-specific human mAb ZKA64-LALA (0.5 μg/ml) (for binding to ZIKV full-length E or EDIII protein), or ZIKV NS2B-NS3-specific mouse sera (for binding to NS2B-NS3 protein). After further washes, the plates were incubated with HRP-conjugated anti-human IgG-Fab (1:3,000) or anti-mouse IgG (1:3,000) antibody at 37° C. for 1 h. The plates were incubated with substrate TMB, and the reaction was stopped by addition of 1 N H₂SO₄. Absorbance at 450 nm (A₄₅₀ values) was measured by an ELISA microplate reader. EC₅₀ values were calculated.

ELISA was also used to determine the ability of ST087010 in inhibition of the binding between ZIKV EDIII and EDIII-specific human mAbs (SMZAb5, ZKA64-LALA, ZV-67, or Z004) or EDI/II-specific human mAb (ZKA78) control. This assay was carried out as described above, except that serially diluted ST087010, gossypol (positive control), or DMSO (negative control) was added to the plates in the presence of each mAb (0.5 μg/mL). The plates were then sequentially incubated with HRP-conjugated anti-human IgG-Fab antibody and TMB substrate, followed by detection of A450 values. Percent inhibition (% inhibition) of compounds was calculated based on the ELISA results, and IC₅₀ was obtained using.

Surface Plasmon Resonance (SPR).

The binding between gossypol derivative ST087010 and ZIKV EDIII or NS2B-NS3 protein was carried out using the Biacore BK system. Briefly, ZIKV EDIII protein or NS2B-NS3 protein were immobilized on a sensor chip (CM5) using Amine Coupling Kit. Serially diluted ST087010 was added as analytes, and HBS-EP with 10% DMSO (10 mM HEPES, 150 mM NaCl, 3 mM EDTA, 0.05% Tween-20, 10% DMSO, pH 7.4) was used as running buffer. Biacore BK evaluation software (version 1.1) was applied to analyze the data, and the curve was fitted with a 1:1 binding model.

Inhibition of NS2B-NS3 Protease Activity.

The ability of gossypol derivative ST087010 in inhibition of NS2B-NS3 protease activity was carried out using a fluorescence-based enzymatic assay. Briefly, serially diluted ST087010, gossypol (positive control), or DMSO (negative control) was incubated with ZIKV NS2B-NS3 protein (1 μg/ml) at 37° C. for 1 h. Substrate (Bz-Nle-Lys-Lys-Arg-AMC, 4 μM) was added, and incubated for 10 min. Fluorescence intensity was then measured at 460 nm (with excitation at 355 nm) using a microplate reader. 10 mM Tris-HCl, 20% glycerol, 1 mM CHAPS, and 5% DMSO (pH 8.5) was used as reaction and dilution buffers. IC₅₀ (concentration at 50% reduction of protease activity) was calculated based on percent inhibition (% inhibition) of compound dilutions.

Protective Efficacy of Gossypol Derivative ST087010 Against ZIKV-Caused Lethal Infection in Ifnar1^{-/-} Mice.

ST087010 was evaluated for protection against ZIKV infection in ZIKV-susceptible Ifnar1^{-/-} mice. Briefly, 7-8-week-old male mice were used, and two separate experiments were performed. In experiment 1, groups of 6 mice were i.p. injected with ST087010 or gossypol (control) (20 mg/kg of body weight), or DMSO (negative control) 12 h before and 6, 24 and 48 h after infection. These mice were infected by i.p. injection with ZIKV (human strain R103451, 200 PFU/mouse), and observed daily for weight changes and survival until 21 days post-infection (dpi). In experiment 2, groups of 5 mice were infected by i.p. injection with ST087010, gossypol, or DMSO as described above, and they were infected by i.p. injection with ZIKV (human strain PAN2016, 200 PFU/mouse). Five days later, these mice were sacrificed, and their tissues, including heart, testis, eye, kidney and brain, collected for detection of viral titers by plaque assay, or assessment of apoptosis by immunofluorescence staining. Mice losing 20% of initial weight with severe symptoms, including hind limb weakness and paralysis, were humanely euthanized.

Plaque assay was used to detect viral titers in ZIKV-infected tissues. Briefly, tissues were homogenized with cold culture medium (DMEM+2% FBS), and then centrifuged (2,000 g at 4° C. for 10 min). Supernatants were serially diluted to infect Vero E6 cells in 24-well plates. ZIKV titers in tissues were measured from ˜40 mg of samples, and thus the detection limit is about 25 PFU/g of tissues.

Protective Efficacy of Gossypol Derivative ST087010 Against ZIKV Vertical Transmission in Pregnant Ifnar1^{-/-} Mice.

ST087010 was evaluated for protection against ZIKV-caused fetal damage and death in pregnant Ifnar1^{-/-} mice. Briefly, groups of 5 pregnant mice (10- to 12-weeks old, E12-14) were injected intraperitoneally with ST087010 (20 mg/kg of body weight), or DMSO (control) 12 h before and 6, 24 and 48 h after i.p. infection with ZIKV (human strain R116265, 10³ PFU). Five days post-infection, mice were sacrificed; viral titers were determined using plaque assay in sera, placenta, fetal brain and amniotic fluid, and uteri and fetuses were evaluated for morphological and size changes. Placenta was also assessed for apoptosis and viral replication by immunofluorescence staining.

Safety of Gossypol Derivative ST087010 in Pregnant Ifnar1^{-/-} Mice and Fetuses.

ST087010 was detected for its safety profiles in pregnant Ifnar1^{-/-} mice and their fetuses. Briefly, groups of 5 pregnant mice (8-12-week-old, E12-14) were i.p. injected with ST087010 (20 or 40 mg/kg of body weight), or DMSO (control), daily for 4 continual days. Mothers and pups at various prenatal and/or postnatal time points were observed for weight changes daily. Sera collected before and 4 h, 1, 3 and 5 days after last injection were measured for ALT and creatinine levels using ALT and Creatinine Assay kits. Two mothers and their pups (at 3-week-old) were sacrificed, and their liver, spleen, kidney, and brain tissues were sectioned, and assessed for histopathological changes by H&E staining.

Protective Efficacy of Gossypol Derivative ST087010 Against DENV Infection in Ifnar1^{-/-} Mice.

ST087010 was evaluated for protection against DENV-2 infection in susceptible Ifnar1^{-/-} mice. Briefly, 3-4-week-old male mice were treated with i.p. administration of ST087010 or gossypol control (20 mg/kg), or DMSO (negative control) 12 h before and 6, 24 and 48 h after infection with DENV-2 human strain (V594, 2×10⁶ PFU/mouse). Three days after infection, sera and tissues, including brain, kidney, and heart, were collected, and detected for DENV infection using a flow cytometry assay (as described below). Tissues were freshly homogenized with cold culture medium (EMEM+2% FBS), and centrifuged at 4° C. and 2,000 g for 10 min. Serially diluted tissue supernatants and sera were added to C6/36 cells (as described below). DENV titers in tissues or sera were determined from ˜20 mg of tissue, or 25 μl of sera, and used for subsequent flow cytometry analysis.

Flow Cytometry Assay.

A flow cytometry assay was carried out to analyze DENV-2 titers in the infected mouse tissue supernatants and sera collected above. Briefly, samples were added to C6/36 cells (5×10⁵ cells/well) seeded in 24-well plates, and incubated for 1 h (28° C., 5% CO₂). After removal of samples and washing with PBS, the cells were incubated with EMEM containing 2% FBS, and cultured for 3 days as above. After further removal of medium, the cells were digested, washed with PBS, and resuspended in FIX/PERM solution, followed by incubation at 4° C. for 1 h in the dark. The cells were then sequentially incubated with mouse anti-flavivirus mAb 4G2 (2 μg/ml) at 37° C. for 1 h, and FITC-labeled anti-mouse IgG (0.5 mg/ml) at 37° C. for 30 min, followed by analysis using FACScan flow cytometer and Summit software. Viral titers (infectious units/ml or infectious units/g) were calculated using the formula: ((% of infected cells)×(total number of cells)×(dilution factor))/(amount of inoculum added to cells).

Immunofluorescence Staining.

Immunofluorescence staining was carried out to detect ZIKV and caspase-3 signals in ZIKV-infected mouse tissues. Briefly, tissues were fixed in 4% formaldehyde, embedded in paraffin, and sectioned. The tissue sections were deparaffinized, fixed, and permed using FIX and PERM Cell Permeabilization Kit. After blocking with 5% BSA, the tissue slides were incubated at 37° C. 2 h with ZIKV EDIII-specific human mAb (ZV-67, 1:100), or rabbit anti-active caspase-3 antibody (1:100). The slides were washed with PBS, and incubated for 30 min with anti-human FITC antibody (1:100, for ZIKV), or anti-rabbit Alexa Fluor 647 antibody (1:100, for caspase-3). The slides were then counter-stained for nuclei using DAPI (300 nM) for 5 min, and mounted in VectaMount Permanent Mounting Medium. The slides were analyzed using a confocal microscope (Zeiss LSM 880) and ZEN software, and fluorescent signals were quantified by ImageJ software.

Statistical Analysis.

The data are presented as mean plus s.e.m. Statistical significances among different groups were analyzed using Student's two-tailed t-test and GraphPad Prism 7 Statistical Software. *, **, and *** indicate P<0.05, P<0.01, and P<0.001, respectively.

Results

Identification of Gossypol Derivatives with Potent Inhibitory Activity Against ZIKV Infection

A series of gossypol derivatives covalently coupled with different chemical groups were detected for their inhibitory activities against ZIKV infection using a plaque-forming assay. Five derivatives showing stronger inhibitory activity than other derivatives, specifically ST069299, ST005138, ST087010, ST092971, and ST086273, were identified as “hit” gossypol derivatives (FIG. 4A). All of the “hit” derivatives were able to effectively inhibit infection of ZIKV (human strain PAN2016), with the IC₅₀ values ranging from 2.29 to 4.98 μM. The cytotoxicity of these derivatives was detected by a cell-based assay in Vero E6 cells, and their CC₅₀ values ranged from 22.82 to 72.13 μM. The cytotoxicity of all five derivatives was reduced compared to gossypol, while ST069299, ST005138, and ST087010 had increased anti-ZIKV inhibitory activity, as compared to gossypol. Structural analysis indicated that all five compounds are derivatives of gossypol with the substitution of C8 and C8′ aldehyde groups, suggesting that the cytotoxicity of gossypol may be related to the C8 and C8′ aldehyde groups, and that replacement of these aldehyde groups in gossypol with other groups resulted in reduced cytotoxicity.

To further analyze the relationship between structure of gossypol and its inhibitory activity, we analyzed the structure of three other gossypol derivatives with significantly reduced inhibitory activity. The results showed that these three compounds are derivatives in which the C7 and CT hydroxyl groups on the gossypol core were substituted, and that their cytotoxicity was also reduced (FIG. 4B). These data suggest that the free hydroxyl groups at the C7 and CT positions on the gossypol core were necessary for gossypol to exert antiviral activity. Thus, replacement of these hydroxyl groups resulted in reduced anti-ZIKV activity of gossypol, as well as decreased cytotoxicity. Structural analysis of these compounds revealed that the aldehyde groups at the C8 and C8′ positions of these three gossypol derivatives were also replaced by other groups, confirming that the cytotoxicity of gossypol is related to the aldehyde groups at the C8 and C8′ positions.

We then evaluated broad-spectrum activity of these gossypol derivatives against nine other ZIKV isolates, such as human strains R116265, PAN2015, FLR, R103451, PRVABC59, PLCal_ZV, and IbH 30656, mosquito strain MEX 2-81, and rhesus macaque strain MR 766, and included gossypol as control. The results showed that, although all gossypol derivatives inhibited infections with the nine ZIKV strains tested with IC₅₀ values at micromolar levels, ST087010 exhibited more potent inhibitory activity than gossypol against seven ZIKV strains tested, and its IC₅₀ values against the ZIKV FLR and R103451 strains were only slightly higher than those of gossypol (Table 7). These data suggest broad-spectrum activity of gossypol derivative ST087010 against multiple strains of ZIKV from different hosts, time periods, and countries.

Selectivity index (SI) was used to evaluate the pharmaceutical safety of these derivatives. In general, the larger the SI value, the higher the safety of drugs. Compared to gossypol, derivative ST087010 had reduced cytotoxicity in Vero E6 cells, with a CC₅₀ that was about 3.5-fold lower than that of gossypol; also, the SI value of ST087010 was much better than that of the other four derivatives with reduced anti-ZIKV activity (FIG. 4A, Table 7). Therefore, ST087010 was identified as the lead gossypol derivative for further studies.

Gossypol Derivative ST087010 Inhibited ZIKV Infection by Targeting the Virus

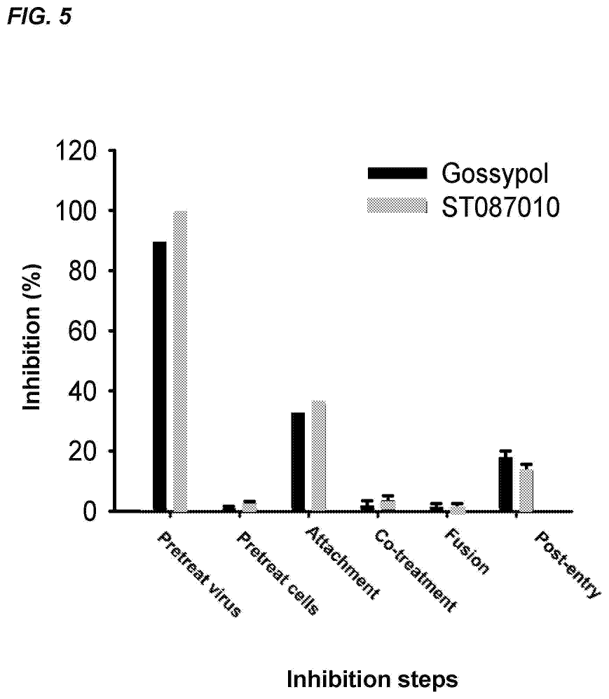

To elucidate the potential inhibitory mechanism of gossypol derivative ST087010 in preventing ZIKV infection. we performed a time-of-addition assay to identify which step of ZIKV life cycle may be interfered. The results showed that ZIKV infection was almost completely inhibited after incubation of the virus with ST087010 at 37° C. for 1 hour, prior to incubation with Vero E6 cells. In contrast, <40% or <20% of ZIKV infection was inhibited, respectively, at viral attachment and post-entry steps, whereas no or little ZIKV infection was blocked in other steps, such as pretreat cells, co-treatment, or fusion steps (FIG. 5). These data suggest that derivative ST087010 inhibited ZIKV infection by mainly targeting the virus, a mechanism very similar to that of gossypol.

TABLE 7
Cytotoxicity and In vitro inhibitory activity of gossypol derivatives against infection
of ZIKV with different strains

| Gossypol | ZIKV strains (IC₅₀: μM) | | | | | |
|---|---|---|---|---|---|---|
| derivatives | PAN2016 | R116265 | PAN2015 | FLR | R103451 | PRVABC59 |
| ST069299 | 2.34 ± 0.06 | 2.57 ± 0.20 | 2.33 ± 0.10 | 3.27 ± 0.25 | 3.85 ± 0.11 | 3.33 ± 0.30 |
| ST005138 | 2.29 ± 0.01 | 3.13 ± 0.29 | 2.18 ± 1.19 | 3.44 ± 0.18 | 3.05 ± 0.09 | 3.89 ± 0.32 |
| ST086273 | 4.81 ± 0.43 | 7.92 ± 0.43 | 4.95 ± 0.17 | 4.20 ± 0.05 | 7.00 ± 0.13 | 4.04 ± 0.22 |
| ST087010 | 3.17 ± 0.18 | 2.91 ± 0.07 | 3.50 ± 0.06 | 2.76 ± 0.10 | 3.60 ± 0.07 | 2.36 ± 0.05 |
| ST092971 | 4.98 ± 0.01 | 7.97 ± 0.17 | 5.01 ± 0.36 | 6.89 ± 0.05 | 6.64 ± 0.27 | 9.10 ± 0.11 |
| Gossypol | 3.78 ± 0.30 | 4.44 ± 0.21 | 4.18 ± 0.04 | 0.73 ± 0.07 | 3.12 ± 0.38 | 4.55 ± 0.07 |

| Gossypol | ZIKV strains (IC₅₀: μM) | | | | |
|---|---|---|---|---|---|
| derivatives | PLCal_ZV | IbH 30656 | MEX 2-81 | MR766 | CC₅₀ (μM) |
| ST069299 | 3.98 ± 0.54 | 2.54 ± 0.44 | 3.89 ± 0.24 | 4.88 ± 0.05 | 25.92 ± 2.70 |
| ST005138 | 3.87 ± 0.54 | 2.40 ± 0.27 | 2.89 ± 0.12 | 2.52 ± 0.04 | 22.82 ± 0.02 |
| ST086273 | 5.87 ± 0.64 | 5.36 ± 0.01 | 2.76 ± 0.01 | 5.46 ± 0.18 | 50.46 ± 3.36 |
| ST087010 | 2.21 ± 0.08 | 2.94 ± 0.08 | 2.92 ± 0.25 | 3.52 ± 0.20 | 49.56 ± 1.83 |
| ST092971 | 6.72 ± 0.20 | 5.16 ± 0.21 | 13.52 ± 0.58 | 5.20 ± 0.37 | 72.13 ± 1.91 |
| Gossypol | 2.88 ± 0.19 | 3.37 ± 0.09 | 2.93 ± 0.01 | 4.14 ± 0.05 | 14.49 ± 0.07 |

The cytotoxicity and inhibitory acitivity of gossypol derivatives against infection of different ZIKV strains were detected in Vero E6 cells. The cytotoxicity is expressed as 50% cytotoxic concentration (CC₅₀). The inhibitory activity of “hit” gossypol derivatives against ZIKV infection is expressed as 50% inhibitory concentration (IC₅₀). Gossypol was used as control. The data are presented as the mean ± standard error of the mean (s.e.m.) (n = 2). The experiments were repeated twice with similar results.

Identification of Binding Region(s) of Gossypol Derivative ST087010 in ZIKV Proteins

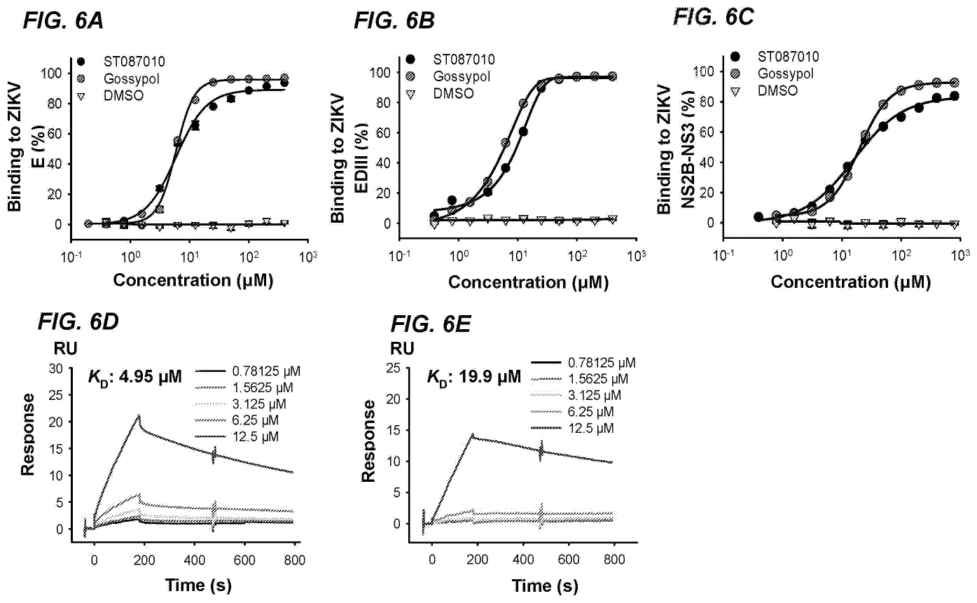

To identify the binding region(s) of gossypol derivative ST087010 in the ZIKV E protein, we performed an ELISA by coating the plate with ZIKV full-length E or EDIII protein, and tested for the binding using ZIKV EDIII-specific mAb ZKA64-LALA. The binding between ST087010 and NS2B-NS3 proteins was performed by coating the ELISA plate with NS2B-NS3 proteins, and detected for the binding using NS2B-NS3 protein-immunized mouse sera. The results showed that ST087010 bound strongly to full-length E, EDIII, and NS2B-NS3 proteins, with EC₅₀ values of 6.47, 6.13, and 21.85 μM, respectively, which were similar to those of gossypol (FIG. 6A-C). Nevertheless, no binding was detected between DMSO control and any of these proteins. Further results from SPR assay revealed that ST087010 bound potently to ZIKV EDIII or NS2B-NS3 protein, with K_{D} (binding affinity) values of 4.95 or 19.9 μM (FIG. 6D-E).

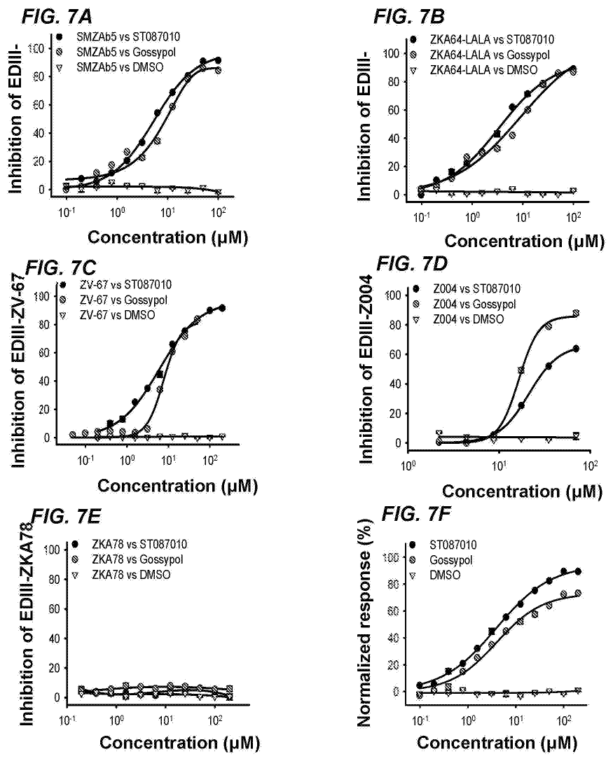

We next identified the potential binding site(s) of ST087010 in the ZIKV EDIII region using an ELISA competition assay. A plate was coated with ZIKV EDIII protein, and the binding of ZIKV EDIII to EDIII-specific neutralizing mAbs, including SMZAb5, ZKA64-LALA, ZV-67, and Z004, was detected in the presence of ST087010 at serial dilutions. The results indicated that similar to gossypol, ST087010 effectively blocked the binding between EDIII and these mAbs dose dependently, resulting in the IC₅₀ values of 5.72, 4.17, 6.70, and 44.51 μM, respectively, for SMZAb5, ZKA64-LALA, ZV-67, or Z004; in contrast, DMSO control had no ability to block the binding of EDIII to any of these mAbs (FIG. 7A-D). As expected, since a ZIKV EDI/II-specific mAb control ZKA78 does not bind to EDIII protein, there was no signal and no observable effect by ST087010 or gossypol control on binding of EDI/II specific antibodies (FIG. 7E). The above EDIII-specific mAbs recognize epitopes on the lateral ridge of ZIKV EDIII protein, suggesting that like gossypol, ST087010 also potentially binds to these epitopes of ZIKV EDIII protein, thus blocking the binding between EDIII and EDIII-specific mAbs.

Since ST087010 bound to ZIKV NS2B-NS3 protein, we wanted to know if it can block the cleavage of this protease. A fluorescence-based inhibition assay was performed in the presence of serially diluted ST087010. The results demonstrated that similar to gossypol, ST087010 indeed inhibited ZIKV NS2B-NS3 protease cleavage in a dose-dependent manner, with an IC₅₀ value of 4.84 μM, whereas DMSO control had no inhibitory activity against this cleavage (FIG. 7F). These results confirmed the ability of ST087010 in strongly inhibiting activity of ZIKV NS2B-NS3 protease.

Gossypol Derivative ST087010 Protected Ifnar1^{-/-} Mice from Lethal ZIKV Challenge and Inhibited Viral Infection

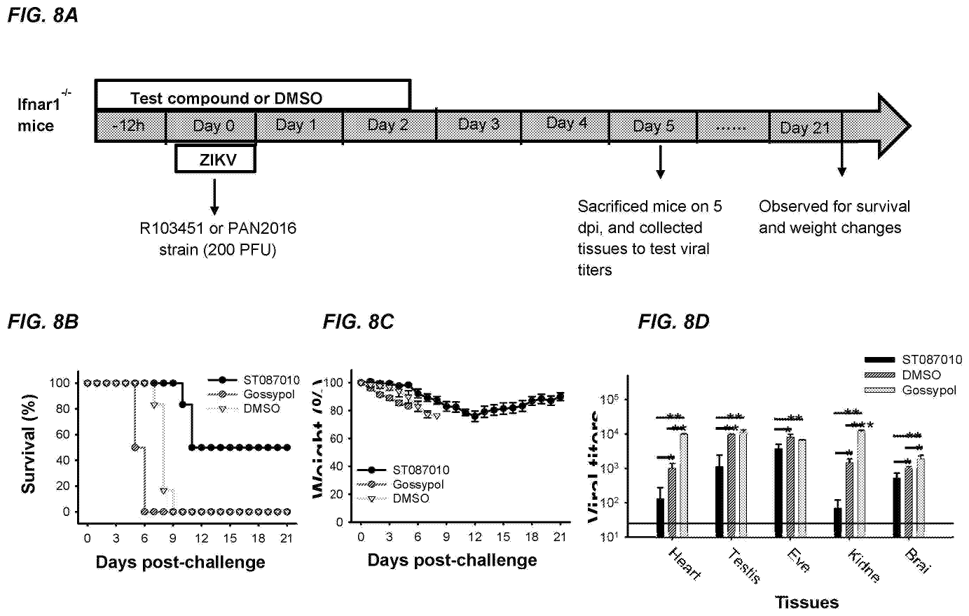

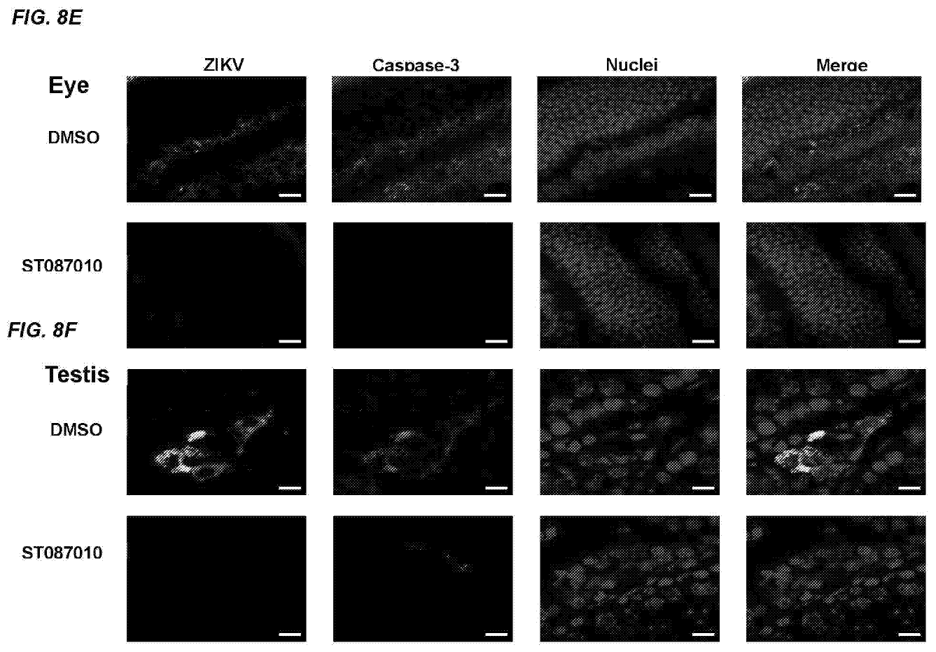

The above in vitro data identified improved anti-ZIKV activity of gossypol derivate ST087010. We next tested the in vivo antiviral effect of ST087010 in adult Ifnar1^{-/-} mice, which are highly susceptible to ZIKV infection. Mice were treated with i.p administration of ST087010 (20 mg/kg), gossypol (20 mg/kg), or DMSO control, 12 h before infection and 6, 24, and 48 h post-infection, and they were infected by i.p. injection of either ZIKV human strain R103451, followed by observation of survival and weight changes for 21 days, or ZIKV human strain PAN2016, followed by detection of ZIKV titers in different tissues at 5 dpi (FIG. 8A).

As shown in FIG. 8B, all mice treated with DMSO died by 9 dpi, whereas treatment with ST087010 protected 50% of the mice from death caused by ZIKV infection. In contrast, no mice treated with gossypol survived past 6 dpi, and these mice even died earlier than DMSO-treated mice, suggesting that the death in the gossypol-treated mice might be due, at least in part, to the toxicity of gossypol itself. In addition, gossypol or DMSO-treated mice showed increased weight loss, whereas the mice treated with ST087010 presented slight weight loss during 6-12 dpi, and then kept steady increase of weight afterwards (FIG. 8C). Moreover, ST087010-treated mice had significantly reduced viral titers in heart, testis, eye, kidney, and brain than gossypol or DMSO-treated mice; but gossypol-treated mice had even significantly higher viral titers in heart, kidney and brain than DMSO-treated mice (FIG. 8D), suggesting that such consequence might be potentially caused by toxicity of gossypol. These data indicate strong activity of gossypol derivative ST087010 in protecting mice against ZIKV-caused death and weight loss and preventing viral replication in challenged mice from two different human strains tested.

To identify potential mechanisms of ST087010 in inhibiting viral infection and ZIKV-caused tissue damage, we stained eye and testicle tissues from ST087010- or DMSO control-treated mice collected at 5 dpi for activated form of caspase-3, an apoptotic marker. The results from immunofluorescence staining indicated that undetectable or diminished staining for caspase-3 was observed in the eye and testis tissues from mice treated with ST087010 as compared to the mice treated with DMSO, which was accompanied by undetectable straining of ZIKV⁺ signals (FIG. 8E-F). These data suggest that gossypol derivative ST087010 prevented ZIKV-caused apoptosis and cell death.

Gossypol Derivative ST087010 Blocked Vertical Transmission of ZIKV in Pregnant Ifnar1^{-/-} Mice, Preventing Fetal Death

ZIKV may vertically transmit from mothers to fetuses, causing fetal damage or death. We tested the efficacy of ST087010 in blocking ZIKV vertical transmission in Ifnar1^{-/-} mice. Pregnant mice (embryonic day (E) 12-14) were injected intraperitoneally with ST087010 (20 mg/kg) or DMSO (control) 12 h before infection and 6, 24 and 48 h post-infection, and they were infected by i.p. administration with another ZIKV human strain (R116265, 10³ PFU), followed by collection of sera and tissues at 5 dpi, and detection of viral titers by plaque assay of sera, placenta, fetal brain, and amniotic fluid, as well as observation of morphological changes in uteri and fetuses, and apoptosis in placentas.

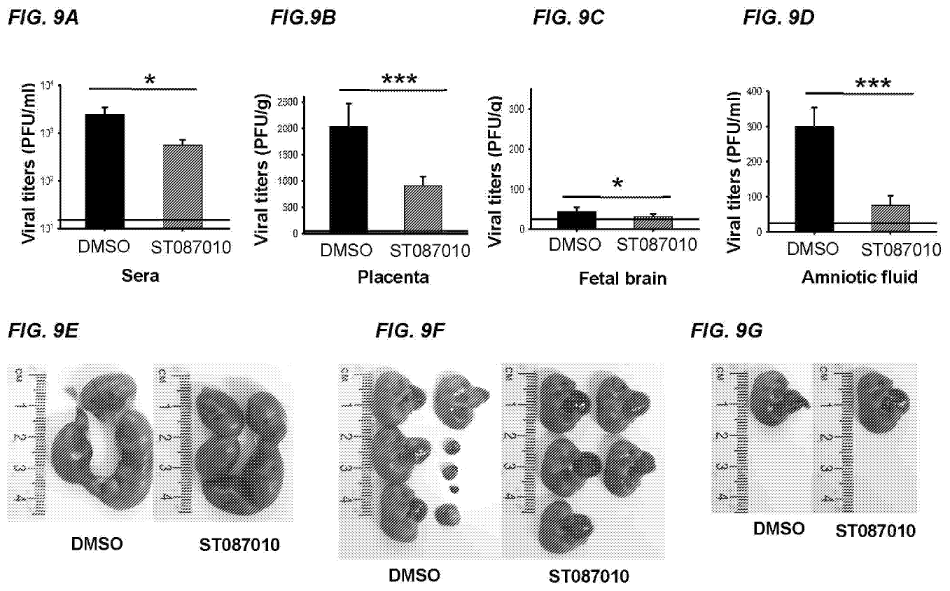

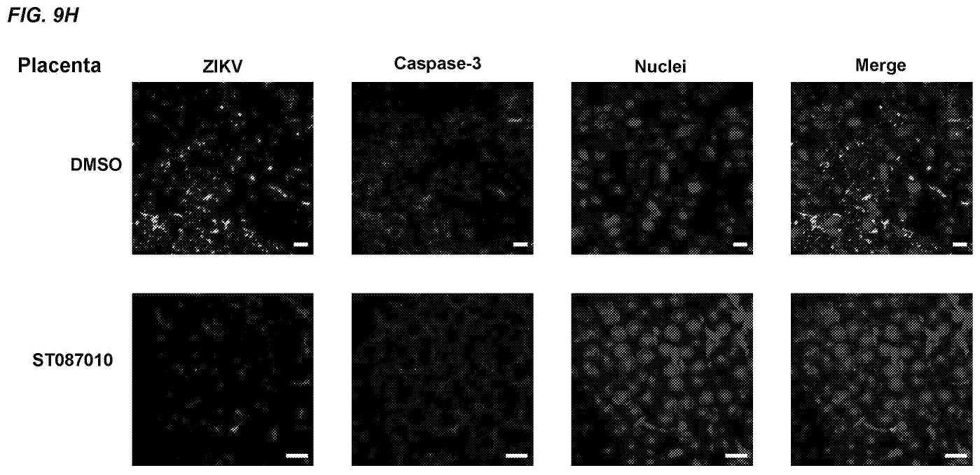

The results showed that viral titers were significantly reduced in ST087010-treated sera (FIG. 9A), placenta (FIG. 9B), fetal brain (FIG. 9C), and amniotic fluid (FIG. 9D) as compared to those of control mice treated with DMSO. In addition, some of the fetuses from DMSO-treated pregnant mice died in uteri, while the fetuses from ST087010-treated mice were all in good condition and their uteri had intact morphology (FIG. 9E-G). Particularly, the size of fetuses treated with DMSO was much smaller than that of the fetuses treated with ST087010 (FIG. 9G), suggesting growth restriction. These data demonstrate that ST087010 prevented vertical transmission of ZIKV from mothers to fetuses, thus preventing ZIKV-caused fetal growth restriction and fetal death.

Immunofluorescence staining of ZIKV⁺ or caspase-3 signals was undetectable or diminished in the placental tissues from mice treated with ST087010, as compared to those from DMSO-treated mice (FIG. 9H), suggesting that ST087010 prevented ZIKV-associated apoptosis and viral replication.

Gossypol Derivative ST087010 was Safe for Pregnant Ifnar1^{-/-} Mice and their Fetuses and Pups

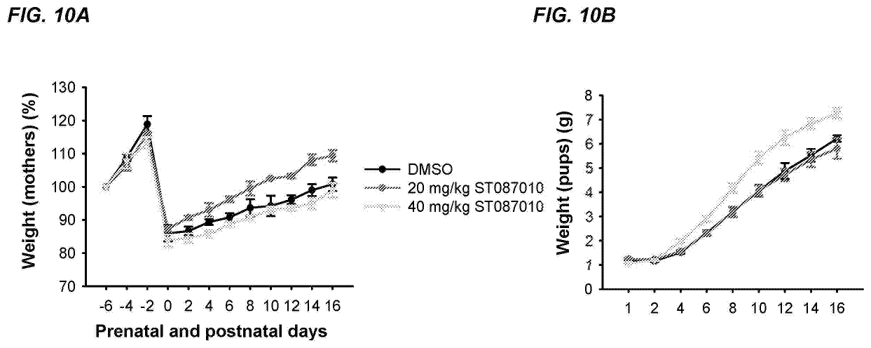

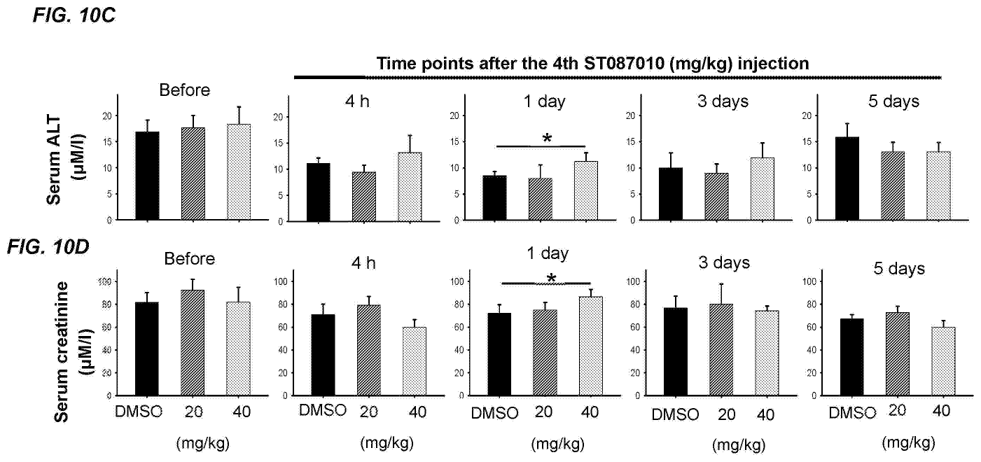

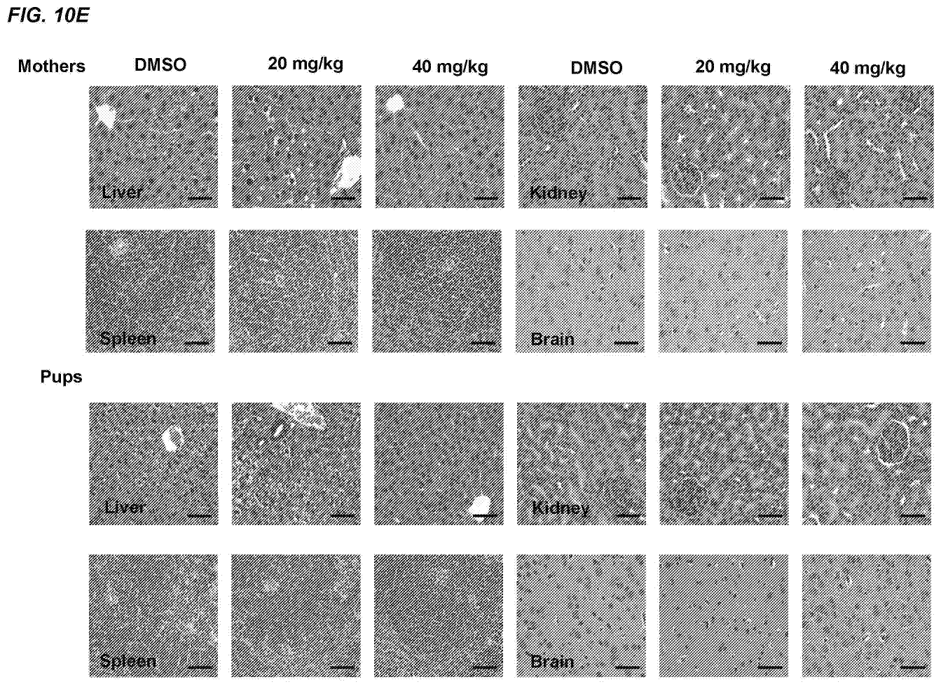

It is important that ZIKV therapeutics should have robust safety for pregnant individuals since the virus causes congenital ZIKV syndrome with significant growth abnormalities to fetuses. Here, we assessed the safety of ST087010 in pregnant Ifnar1^{-/-} mice. The results showed that pregnant mice (FIG. 10A) and their pups (FIG. 10B) had similar weight, or only small weight changes, after the mice received ST087010 at 20 or 40 mg/kg, or DMSO control, suggesting that ST087010 did not result in significant damage to the pregnant mice and their fetuses, and thus pups grew normally.

We also measured alanine aminotransferase (ALT) (FIG. 10C) and creatinine (FIG. 10D) levels in the sera of mice prior to and after receiving ST087010 or DMSO at different time points. The results showed no significant differences of ALT and creatinine levels in the mice before injection and 4 h, 3 or 5 days after last injection of ST087010, suggesting that injection of pregnant mice with ST087010 at 20 or 40 mg/kg did not change their hepatic and renal function. Although serum ALT and creatinine levels were significantly different between high-dose ST087010-treated mice (40 mg/kg) and DMSO-treated mice at 1 day after injection, these elevations were transient. Moreover, there was no significant difference between the two groups for the mice receiving low-dose ST087010 (20 mg/kg), a dose which showed strong anti-ZIKV activity in vivo.

Histopathological analysis of DAPI-stained tissues from mothers and pups indicated that liver, spleen, kidney, and brain tissue of mice treated with ST087010 at 20 or 40 mg/kg presented no abnormal pathological changes, as compared to those of mice treated with DMSO (FIG. 10E). In addition, no inflammation or cell infiltration was observed in the mice treated with ST087010 at either low and high doses, suggesting that gossypol derivative ST087010 was safe for pregnant mice and their fetuses, even at the high dose of 40 mg/kg.

Potent In Vitro Inhibitory Activity of Gossypol Derivative ST087010 Against Infection of DENV-1-4 Strains

We further detected broad-spectrum activity of gossypol derivative ST087010 against infection of other flaviviruses, such as DENV, and compared the results with those of gossypol. As such, human strains of the four serotypes of DENV, DENV-1-V1792, DENV-2-V594, DENV-3-V1043, and DENV-1-PR 06-65-740, were tested by plaque assay for inhibition of viral infection of LLC-MK2 cells by ST087010. The results showed that although ST087010 had slightly higher IC₅₀ values than gossypol against infection of the DENV-1, DENV-2, and DENV-4 strains tested, its cytotoxicity was much lower than that of gossypol, with a CC₅₀ value of 47.2 μM (Table 8). By comparing inhibitory activity and cytotoxicity profiles, we found that ST087010 had SI values of 15.7, 14.8, 14.2, and 15.8, respectively, against infection of DENV-1, DENV-2, DENV-3 and DENV-4, which were much higher than those of gossypol. These data suggest strong broad-spectrum activity of ST087010 against DENV-1-4 infection in vitro with lower cytotoxicity and higher safety, as compared to gossypol.

TABLE 8
In vitro inhibitory activity of gossypol derivative ST087010 against infection of DENV-1-4 strains

| | | DENV-1-4 strains (IC_{50:} μM) | | | | | | | |
|---|---|---|---|---|---|---|---|---|---|
| Gossypol | | DENV-1- | | DENV-2- | | DENV-3- | | DENV-4- | |
| derivative | CC₅₀ (μM) | V1792 | SI | V594 | SI | V1043 | SI | PR 06-65-740 | SI |
| ST087010 | 47.20 ± 1.35 | 3.01 ± 0.11 | 15.7 | 3.19 ± 0.01 | 14.8 | 3.33 ± 0.08 | 14.2 | 2.98 ± 0.18 | 15.8 |
| Gossypol | 19.06 ± 1.09 | 2.06 ± 0.13 | 9.3 | 1.91 ± 0.01 | 10.0 | 3.42 ± 0.11 | 5.6 | 2.83 ± 0.07 | 6.7 |

Note:
The cytotoxicity and inhibitory activity of gossypol derivative ST087010 were detected in LLC-MK2 cells, and gossypol was included as control. The cytotoxicity is expressed as CC₅₀. The inhibitory activity against DENV-1-4 infection is expressed as IC₅₀. Selectivity index (SI) was calculated based on the values CC₅₀/IC₅₀. The data are presented as mean ± s.e.m. (n = 2). The experiments were repeated twice with similar results.

Gossypol Derivative ST087010 Inhibited DENV-2 Replication in Ifnar1^{-/-} Mice

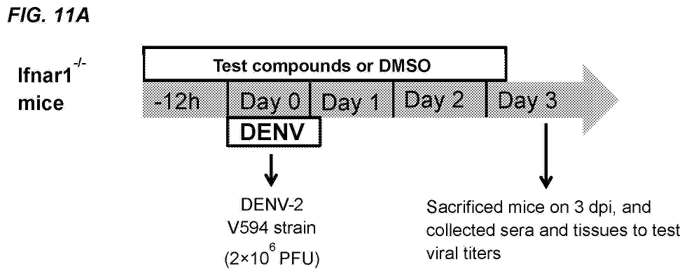

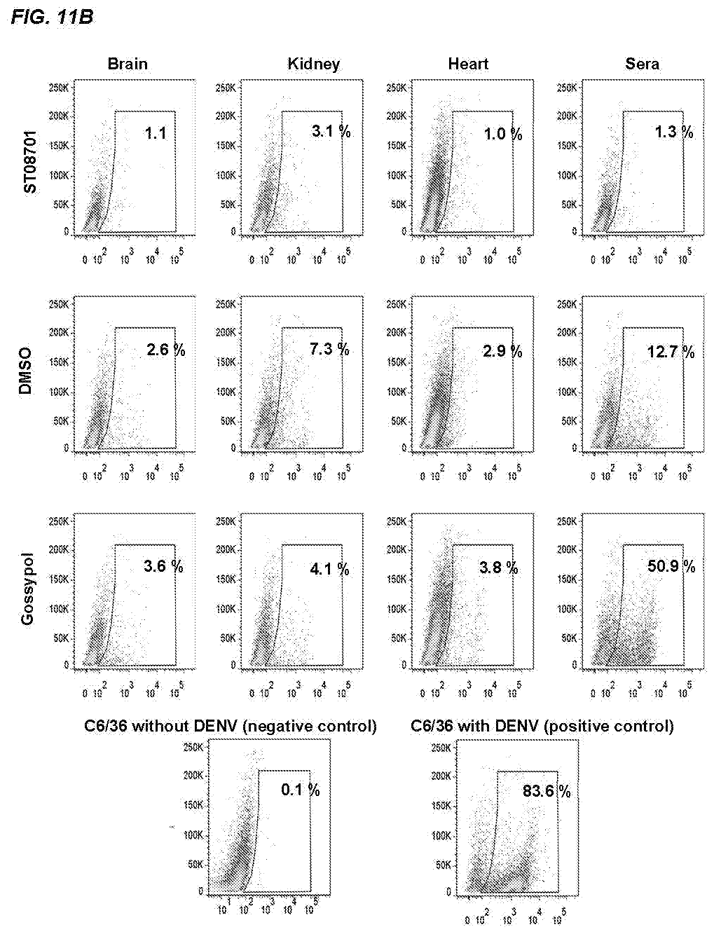

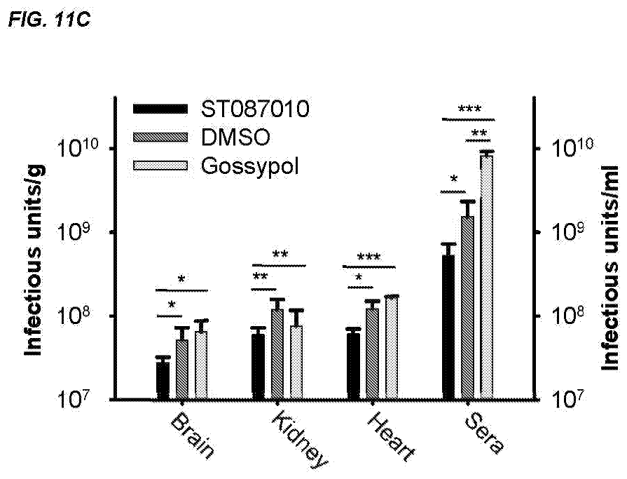

To detect the ability of ST087010 to prevent DENV infection in vivo, 3- to 4-week-old Ifnar1^{-/-} mice were administered ST087010 (20 mg/kg), gossypol (20 mg/kg), or DMSO, by i.p. injection 12 h before and 6, 24 and 48 h after infection. The mice were infected by i.p. administration of DENV-2 (human strain V594, 2×10⁶ PFU), and viral loads measured (based on the number of virus-infected cells) in tissues (brain, kidney, and heart) and sera collected at 3 dpi (FIG. 11A). C6/36 cells were infected with supernatant of tissue samples and sera, and the number of infected cells was determined by a flow cytometry-based assay. The results revealed that mice treated with ST087010 had a significantly reduced number of infected cells (viral titer) in brain, kidney, heart, and sera, as compared to those treated with gossypol and DMSO (FIG. 11B-C), suggesting inhibition of DENV replication in the ST087010-treated mice. These data demonstrate broad-spectrum inhibitory activity of gossypol derivative ST087010 against other flaviviruses, such as DENV.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified, thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating or inhibiting a flavivirus infection, comprising administering an effective amount of one or more of gossypol, digitonin, conessine, ST069299, ST005138, ST092971, ST086276, or ST087010 to a person in need thereof.

2. (canceled)

3. (canceled)

4. The method of claim 1, wherein the administering takes place within 12 hours prior to potential exposure.

5. The method of claim 1, wherein the administering takes place within 24 hours after exposure or potential exposure.

6. (canceled)

7. The method of claim 1, comprising administration of one or more of gossypol, ST069299, ST005138, ST092971, ST086276, or ST087010.

8. The method of claim 7, further comprising administration of digitonin or conessine.

9. The method of claim 1, comprising administration of ST069299.

10. The method of claim 9, further comprising administration of digitonin or conessine.

11. The method of claim 1, comprising administration of ST005138.

12. The method of claim 11, further comprising administration of digitonin or conessine.

13. The method of claim 1, comprising administration of ST092971.

14. The method of claim 13, further comprising administration of digitonin or conessine.

15. The method of claim 1, comprising administration of ST086276.

16. The method of claim 15, further comprising administration of digitonin or conessine.

17. The method of claim 1, comprising administration of ST087010.

18. The method of claim 17, further comprising administration of digitonin or conessine.

19. (canceled)

20. (canceled)

21. (canceled)

22. (canceled)

23. The method of claim 1, further comprising administration of an effective amount of curcumin or bortezomib.

24. (canceled)

25. The method of claim 1, wherein the flavivirus is a Spondweni virus, a dengue virus, a Japanese encephalitis group virus, a yellow fever group virus.

26. The method of claim 25, wherein the Spondweni virus is Zika virus.

27. (canceled)

28. (canceled)

29. (canceled)

30. (canceled)

31. The method of claim 1, wherein the person in need thereof is infected with the flavivirus.

32. The method of claim 1, wherein the person in need thereof has been exposed to the flavivirus.

33. The method of claim 1, wherein the person in need thereof is at risk of exposure to the flavivirus.
